(19) United States
(12) Patent Application Publication
(10) Pub. No.: US 2012/0052577 A1
(43) Pub. Date: Mar. 1, 2012

(54) CULTURE SYSTEM FOR STEM CELL PROPAGATION AND NEURAL AND OLIGODENDROCYTE SPECIFICATION

(75) Inventors: Maria Dolores Araceli Espinosa de los Monteros, Marina del Rey, CA (US); Jean S. de Vellis, Pacific Palisades, CA (US)

(73) Assignee: THE REGENTS OF THE UNIVERSITY OF CALIFORNIA, Oakland, CA (US)

(21) Appl. No.: 13/223,164

(22) Filed: Aug. 31, 2011

Related U.S. Application Data
(60) Provisional application No. 61/402,598, filed on Aug. 31, 2010.

(51) Int. Cl. C12N 5/079; C12N 5/071

(52) U.S. Cl. 435/377; 435/406

(57) ABSTRACT

The present invention provides methods and compositions for culturing stem cells, such as neural stem cells, and includes inducing the specification of neural stem cells to the oligodendrocyte phenotype and specification of multipotent cells ie. iPS cells and ES cells.

### CROSS-REFERENCE TO RELATED APPLICATIONS

This application claims the benefit of U.S. provisional application No. 61/402,598, filed Aug. 31, 2010.

### STATEMENT AS TO RIGHTS TO INVENTIONS MADE UNDER FEDERALLY SPONSORED RESEARCH AND DEVELOPMENT

This invention was made with government support under grant no. PPG HD006576, awarded by the National Institutes of Health. The Government has certain rights in the invention.

### FIELD OF THE INVENTION

The present invention relates generally to the area of stem cell culture. In particular, the invention relates to methods and compositions for culturing neural stem cells and inducing their specification to the oligodendrocyte phenotype.

### BACKGROUND OF THE INVENTION

We have previously designed and routinely used several culture media to propagate and maintain neural stem cells (NSC), as well as cell culture media that we have designed to induce the specification of NSC to the oligodendrocyte (OL) phenotype. These two media formulas used in a specific manner in combination with previously described culture media for OL maturation, “GDM” and “OLDEM” (a nutrient formula that supports OL complete development to reach their functional myelinating stage), have resulted in a culture system for the generation of neural stem cells and OL (Espinosa-Jeffrey et al., “Selective Specification of CNS Stem Cells Into Oligodendroglial or Neuronal Cell Lineage: Cell Culture and Transplant Studies,” J. Neuroscience Res. 69:810-25 (2002) (which is hereby incorporated by reference in its entirety and specifically for its description of methods and compositions for generating neural stem cells and OL); Espinosa-Jeffrey et al., “Culture System for Rodent and Human Oligodendrocyte Specification, Lineage Progression, and Maturation,” Curr. Protocols in Stem Cell Biol. 2D.4.1-2D.4.26 (2009) (which is hereby incorporated by reference in its entirety and specifically for its description of methods and compositions for generating neural stem cells and OL).

### BRIEF DESCRIPTION OF THE DRAWINGS

FIG. 1. Oligodendrocyte specification and lineage progression. Oligodendrocytes undergo sequential morphological changes as they develop from uncommitted NSC to a committed OLP and acquire characteristics inherent in a functional OL. The list of OL markers below each developmental stage is not exhaustive but represents frequently used markers to identify OLs and their developmental stage. We used Tf, PDGF alpha receptor, Sulfatides and MBP as indicators of the developmental stages of cells cultured in 0G condition. Media used: (also see methods) stem cell medium (STM; Espinosa et al., 2002); OL specification medium (OSM; Espinosa et al., 2002); glia defined medium (GDM; Espinosa et al., 1994); OLDEM (Espinosa et al., 1988, 1997). Chart modified from Arenander and de Vellis (1995).

FIG. 2. The expression of neural stem cell markers by the neural stem cell line 2050 cultured, as described in Example 2, in STM for 1 month or 1 day (lanes 1 and 2) and hIPS-21 cultured 1 month in iP/ESSOLM lane 3.

FIG. 3. Cultures produced as described in Example 2 were examined to determine that these cells expressed oligodendrocyte-specific markers using triple immunofluorescence for neural (Pax 6 and nestin) and oligodendrocyte markers such as Olig2, sulfatides detected by the antibody O4, and galactocerebrosides (GC). These cultures did not contain astrocytes as shown by the absence of glial fibrilary acidic protein (GFAP).

FIG. 4. Oligodendrocyte specification and lineage progression, illustrating the use of a Medium for Specification of Multipotent (iPS/ES) Stem Cells to Oligodendrocyte Progenitors (iP/ESS-OLM), described in Table 5. See Example 2.

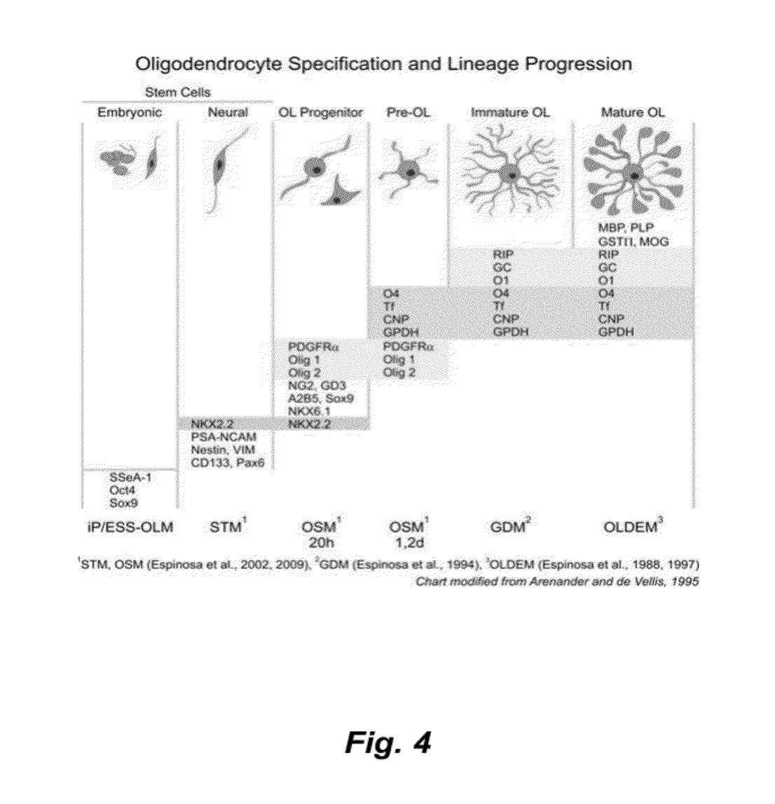

### DETAILED DESCRIPTION

The present invention includes novel nutrient formulas that can be used in a stem cell medium (STM) and an OL transition medium, now termed “oligodendrocyte specification medium (OSM),” which is useful in a culture system for the generation of generation of neural stem cells and OL. The invention also includes media and cultures containing the nutrient formulas. The culture system described herein for the generation of neural stem cells and OL has proven to work with mouse, rat, non-human primate, and human neural stem cells (NSC), allowing for the generation of OL progenitors as well as mature myelinating cells. In certain embodiments, the invention extends further to provide the specification of multipotent stem cells, i.e., induced pluripotent stem cells and embryonic stem cells to oligodendrocytes.

The methods and compositions described herein afford the following advantages with respect to existing stem cell sources for cell replacement therapies (e.g., for treatment of brain and/or spinal cord injury, myelin disorders, and many neurodegenerative disorders): cells can be maintained in an undifferentiated state if desired to increase their numbers, and these cells can be induced to a desired neural phenotype, minimizing the presence of undesirable cell types, providing a better source for transplants. Thus, cells generated using the methods and compositions described here are therapeutically relevant and commercially valuable. In addition, these methods and compositions can be used in producing autologous NSC and their derivatives, i.e., oligodendrocytes, neurons and astrocytes for autologous grafting.

### DEFINITIONS

Terms used in the claims and specification are defined as set forth below unless otherwise specified.

As used herein, the term “stem cell” refers to a cell that has the capacity for self-renewal, i.e., the ability to go through numerous cycles of cell division while maintaining the undifferentiated state. Stem cells can be totipotent, pluripotent, multipotent, oligopotent, or unipotent. Stem cells can be embryonic, fetal, amniotic, adult, or induced pluripotent stem cells.

As used herein, the term “induced pluripotent stem cells” refers to a type of pluripotent stem cell that is artificially derived from a non-pluripotent cell, typically an adult somatic cell, by inducing expression of specific genes.

As used herein, the term “neural stem cell (NSC)” refers to a cell that is capable of giving rise only to the following central nervous system (CNS) cells: neurons, astrocytes, and oligodendrocytes/oligodendroglial cells. However, NSCs maintain the ability to self-renew.

As used herein, the term “mesenchymal stem cell (MSC) refers to a multipotent stem cell that can differentiate into a variety of cell types, including osteoblasts (bone cells), chondrocytes (cartilage cells) and adipocytes (fat cells).

### Nutrient Formulas

In certain embodiments, the invention includes two nutrient formulas. One nutrient formula is useful in propagating stem cells, e.g., neural stem cells (NSC). This formula, which is typically included in a stem cell medium (see Table 3, below), is shown in Table 1.

TABLE 1
Nutrient Formula for Stem Cell Medium

| | Molarity | | |
|---|---|---|---|
| Components | (mM) | mg/L | |
| Hydroxybutyrate | 0.5 | 80.1 | |
| Hypoxanthine-Na | 0.0075 | 1.192 | |
| i-Inositol | 0.055 | 9.9 | |
| L-Alanine | 0.025 | 2.225 | |
| L-Arginine hydrochloride | 0.549 | 115.839 | |
| L-Asparagine-H2O | 0.025 | 3.75 | |
| L-Cysteine-HCl—H2O | 0.05 | 8.8 | |
| L-Cystine-2HCl | 0.15 | 46.95 | |
| L-Glutamine | 2 | 292 | |
| L-Histidine-HCl—H2O | 0.175 | 36.75 | |
| L-Isoleucine | 0.609 | 79.779 | |
| L-Leucine | 0.6265 | 82.0715 | |
| L-Lysine hydrochloride | 0.6485 | 118.6755 | |
| L-Methionine | 0.1585 | 23.6165 | |
| L-Phenylalanine | 0.3075 | 50.7375 | |
| L-Proline | 0.075 | 8.625 | |
| L-Serine | 0.325 | 34.125 | |
| L-Threonine | 0.2635 | 31.3565 | |
| L-Tryptophan | 0.0611 | 12.4644 | |
| L-Tyrosine-2Na—2H2O | 0.306 | 68.85 | |
| L-Valine | 0.6275 | 73.4175 | |
| Lipoic acid | 0.000255 | 0.05253 | |
| Creatine | 0.0128 | 1000 | mg/L |
| bFGF | | 20 | ng/ml |
| EGF | | 10 | ng/ml |
| Insulin | | 10 | μg/ml |
| Transferrin | | 50 | mg/L |
| Pen/Strep | | 0.5 | ml/L |
| B 27 with Retinoic acid* | | 1 | ml/L |

*B-27 with Retinoic acid is included in particular embodiments.

Insulin-like growth factor 1 (IGF-1, e.g., recombinant) can be added to the Stem Cell Medium to increase stem cell propagation (proliferation) when starting the culture. Illustrative doses range from 0.5 nanograms per milliliter (0.5 ng/ml) to 500 ng/ml, and optimal doses will vary depending on the source of IGF-1. Alternatively, insulin-like growth factor 2 (IGF-2) or insulin within the same range would produce similar results, with higher doses generally being more effective. Optimal concentrations can be determined by the user for all three molecules.

Another nutrient formula is useful in inducing the specification of neural NSC to the oligodendrocyte (OL) phenotype. This formula, which is typically included in an oligodendrocyte specification medium (OSM; previously termed OL transition medium) (see Table 4, below), is shown in Table 2.

TABLE 2
Nutrient Formula for Oligodendrocyte Specification Medium (OSM)

| | | | | Altered | |
|---|---|---|---|---|---|
| | Molarity | Formula | | Concentrations* | |
| Components | (mM) | Weight | mg/L | mg/L | |
| Hydroxy- | 0.25 | | 0 | 35.5 | |
| butyrate | | | | | |
| Hypoxanthine- | 0.01125 | | 0 | 1.788 | |
| Na | | | | | |
| i-Inositol | 0.0625 | 180 | 11.25 | | |
| L-Alanine | 0.0375 | 89 | 3.3375 | | |
| L-Arginine | 0.624 | 211 | 131.664 | | |
| hydrochloride | | | | | |
| L-Asparagine- | 0.0375 | 150 | 5.625 | | |
| H2O | | | | | |
| L-Aspartic acid | 0.025 | 133 | 3.325 | | |
| L-Cysteine- | 0.075 | 176 | 13.2 | | |
| HCl—H2O | | | | | |
| L-Cystine- | 0.125 | 313 | 39.125 | | |
| 2HCl | | | | | |
| L-Glutamic | 0.025 | 147 | 3.675 | | |
| Acid | | | | | |
| L-Glutamine | 2.25 | 146 | 328.5 | | |
| L-Histidine- | 0.16245 | 210 | 34.1145 | | |
| HCl—H2O | | | | | |
| L-Isoleucine | 0.5125 | 131 | 67.1375 | | |
| L-Leucine | 0.52075 | 131 | 68.21825 | | |
| L-Lysine | 0.5735 | 183 | 104.9505 | | |
| hydrochloride | | | | | |
| L-Methionine | 0.137 | 149 | 20.413 | | |
| L- | 0.26125 | 165 | 43.10625 | | |
| Phenylalanine | | | | | |
| L-Proline | 0.1125 | 115 | 12.9375 | | |
| L-Serine | 0.2875 | 105 | 30.1875 | | |
| L-Threonine | 0.17625 | 119 | 20.97375 | 42.3937 | |
| L-Tryptophan | 0.05255 | 204 | 10.7202 | | |
| L-Tyrosine- | 0.26 | 225 | 58.5 | | |
| 2Na—2H2O | | | | | |
| L-Valine | 0.5395 | 117 | 63.1215 | | |
| Linoleic acid | 0.000075 | 280 | 0.021 | | |
| Lipoic acid | 0.00037025 | 206 | 0.076272 | 0.078795 | |
| Creatine | 0.0064 | | 0.860 | 500.00 | |
| B-27 | 0.038194444 | | | 500 | μl/L |
| Transferrin | | | 25 | 50 | |

*This column shows altered concentrations (in mg/L only) for certain components that are used in combination with the other components (at the disclosed concentrations) for particular embodiments.

Insulin-like growth factor 1 (IGF-1, e.g., recombinant) can be added to the Oligodendrocyte Specification Medium to increase stem cell propagation just prior to commitment of these cells. Illustrative doses range from 0.5 nanograms per milliliter (0.5 ng/ml) to 500 ng/ml, and optimal doses will vary depending on the source of IGF-1. Alternatively, insulin-like growth factor 2 (IGF-2) or insulin within the same range would produce similar results, with higher doses generally being more effective. Optimal concentrations can be determined by the user for all three molecules.

When the goal is to maximize oligodendrocyte specification and subsequent proliferation of committed oligodendrocytes progenitors, insulin growth factor 1 (IGF-1)+transferrin (apo-transferrin or Na-transferrin) can be used in combination as additives to the Oligodendrocyte Specification Medium. Illustrative doses for IGF-1 range from 0.5 ng/ml to 500 ng/ml, depending on the source of the IGF-1, and illustrative doses for human (recombinant) transferrin (TO range from 1 microgram/ml (1 μg/ml) to 100 μg/ml, depending on the source of the iron carrier protein.

The invention also includes embodiments in which one or more of the components in Tables 1 or 2 is employed at a concentration that differs from that shown by 0.5%, 1.0%, 1.5%, 2%, 2.5%, 3.0%, 3.5%, 4.0%, 4.5%, or 0.5% or by a percentage falling within a range having any of these values as endpoints (e.g., a percentage falling within the range of 1.0%-2.5%, e.g., 2.3%). The concentration of the component may be higher or lower than that shown in Tables 1 or 2.

### Cell Culture Media

In other embodiments, the invention provides two cell culture media, each containing one of the nutrient formulas described above. The composition of the Stem Cell Medium is given in Table 3.

TABLE 3
Stem Cell Medium

| | | | | | Altered | |
|---|---|---|---|---|---|---|
| | Molarity | Formula | | | Concentrations* | |
| Components | (mM) | Weight | mg/L | | mg/L | |
| Biotin | 0.000011075 | 244 | 0.002702 | | | |
| Calcium chloride | 1.14975 | 111 | 127.6223 | | 138.75 | |
| Choline chloride | 0.0552 | 140 | 7.728 | | | |
| Cupric sulfate | 0.0000038 | 250 | 0.00095 | | | |
| D-Calcium | 0.00555 | 477 | 2.64735 | | | |
| pantothenate | | | | | | |
| D-Glucose | 17 | 180 | 3060 | | 2970 | |
| Ethanolamine | 0.125 | 61 | 7.625 | | | |
| Ferric nitrate | 0.000154 | 404 | 0.062216 | | | |
| Ferrous sulfate | 0.001125 | 278 | 0.31275 | | | |
| Folic acid | 0.00689 | 441 | 3.03849 | | | |
| Fumaric acid | 0.25 | | 0 | | | |
| Glycine | 0.26835 | 75 | 20.12625 | | 21.54 | |
| Hepes | 3.75 | | 0 | | 1785 | |
| Hydroxybutyrate | 0.25 | | 0 | | 80.1 | |
| Hypoxanthine-Na | 0.01125 | | 0 | | 1.192 | |
| i-Inositol | 0.0625 | 180 | 11.25 | | 9.9 | |
| L-Alanine | 0.0375 | 89 | 3.3375 | | | |
| L-Arginine | 0.624 | 211 | 131.664 | | | |
| hydrochloride | | | | | | |
| L-Asparagine-H2O | 0.0375 | 150 | 5.625 | | | |
| L-Aspartic acid | 0.025 | 133 | 3.325 | | | |
| L-Cysteine-HCl—H2O | 0.075 | 176 | 13.2 | | | |
| L-Cystine-2HCl | 0.125 | 313 | 39.125 | | | |
| L-Glutamic Acid | 0.025 | 147 | 3.675 | | | |
| L-Glutamine | 2.25 | 146 | 328.5 | | | |
| L-Histidine-HCl—H2O | 0.16245 | 210 | 34.1145 | | | |
| L-Isoleucine | 0.5125 | 131 | 67.1375 | | | |
| L-Leucine | 0.52075 | 131 | 68.21825 | | | |
| L-Lysine | 0.5735 | 183 | 104.9505 | | | |
| hydrochloride | | | | | | |
| L-Methionine | 0.137 | 149 | 20.413 | | | |
| L-Phenylalanine | 0.26125 | 165 | 43.10625 | | | |
| L-Proline | 0.1125 | 115 | 12.9375 | | | |
| L-Serine | 0.2875 | 105 | 30.1875 | | | |
| L-Threonine | 0.17625 | 119 | 20.97375 | | | |
| L-Tryptophan | 0.05255 | 204 | 10.7202 | | | |
| L-Tyrosine-2Na—2H2O | 0.26 | 225 | 58.5 | | | |
| L-Valine | 0.5395 | 117 | 63.1215 | | | |
| Linoleic acid | 0.000075 | 280 | 0.021 | | | |
| Lipoic acid | 0.00037025 | 206 | 0.076272 | | | |
| Magnesium chloride | 0.225 | 95 | 21.375 | | | |
| Magnesium sulfate | 0.50825 | 120 | 60.99 | | | |
| Niacinamide | 0.0206 | 122 | 2.5132 | | | |
| Phenol red | 0.01805 | 398 | 7.1839 | | 6.8854 | |
| Potassium chloride | 4.435 | 75 | 332.625 | | | |
| Putrescine-2HCl | 0.00037575 | 161 | 0.060496 | | | |
| Pyridoxine | 0.0123 | 206 | 2.5338 | | | |
| hydrochloride | | | | | | |
| Pyruvic acid | 0.25 | | 0 | | | |
| Riboflavin | 0.00070525 | 376 | 0.265174 | | | |
| Sodium chloride | 118.085 | 58 | 6848.93 | | | |
| Sodium bicarbonate | 36.375 | 84 | 3055.5 | | 3670.8 | |
| Sodium phosphate, | 0.33975 | | 0 | | 96.489 | |
| mono. | | | | | | |
| Sodium phosphate, | 0.125 | | 0 | | 34.50 | |
| dibas. | | | | | | |
| Sodium pyruvate | 1.125 | 110 | 123.75 | | 137.5 | |
| Thiamine | 0.00775 | 337 | 2.61175 | | 3.00667 | |
| hydrochloride | | | | | | |
| Thymidine | 0.001125 | 242 | 0.27225 | | | |
| Vitamin B12 | 0.000625 | 1355 | 0.846875 | | | |
| Zinc sulfate | 0.001125 | 288 | 0.324 | | | |
| Creatine | 3.355 | 131.13 | 2000 | | 1000 | |
| B-27+ Retinoic acid** | 0.038194444 | | | | 1 | ml/L |
| bFGF | | | | | 20 | ng/ml |
| EGF | | | | | 10 | ng/ml |
| Kanamycin | | | 1 | ml/L | | |
| Insulin | | | 5 | | 10 | |
| Transferrin | | | 50 | | 100 | |
| Pen/Strep | | | | | 1 | ml/L |
| Normocin*** | | | | | 1 | ml/L |

*This column shows altered concentrations (in mg/L only) for certain components that are used in combination with the other components (at the disclosed concentrations) for particular embodiments.
**Retinoic acid is included in the B-27 in particular embodiments.
***Normocin is included in particular embodiments.
Creatine source: Creatine Hydrate minimum 99%
Sigma C-3630
H20 content 0.8 mol/mol

The composition of the oligodendrocyte specification medium (OSM; previously termed OL transition medium) is given in Table 4.

TABLE 4
Oligodendrocyte Specification Medium (OSM)

| | | | | Altered |
|---|---|---|---|---|
| | Molarity | | | Concentrations* |
| Components | (mM) | Formula Weight | mg/L | mg/L |
| Biotin | 0.000011075 | 244 | 0.002702 | |
| Calcium chloride | 1.14975 | 111 | 127.6223 | 137.3625 |
| Choline chloride | 0.0552 | 140 | 7.728 | |
| Cupric sulfate | 0.0000038 | 250 | 0.00095 | 3735 |
| D-Calcium | 0.00555 | 477 | 2.64735 | |
| pantothenate | | | | |
| D-Glucose | 17 | 180 | 3060 | |
| D-Galactose | | | 2300 | |
| Ethanolamine | 0.125 | 61 | 7.625 | 7.625 |
| Ferric nitrate | 0.000154 | 404 | 0.062216 | |
| Ferrous sulfate | 0.001125 | 278 | 0.31275 | |
| Folic acid | 0.00689 | 441 | 3.03849 | |
| Fumaric acid | 0.25 | | 34.5 | 34.5 |
| Glycine | 0.26835 | 75 | 20.12625 | 30.9 |
| Hepes | 3.75 | | 0 | 892.5 |
| Hydroxybutyrate | 0.25 | | 35.5 | 40 |
| Hypoxanthine-Na | 0.01125 | | 1.78875 | |
| i-Inositol | 0.0625 | 180 | 11.25 | |
| L-Alanine | 0.0375 | 89 | 3.3375 | |
| L-Arginine | 0.624 | 211 | 131.664 | |
| hydrochloride | | | | |
| L-Asparagine-H2O | 0.0375 | 150 | 5.625 | |
| L-Aspartic acid | 0.025 | 133 | 3.325 | |
| L-Cysteine-HCl—H2O | 0.075 | 176 | 13.2 | |
| L-Cystine-2HCl | 0.125 | 313 | 39.125 | |
| L-Glutamic Acid | 0.025 | 147 | 3.675 | |
| L-Glutamine | 2.25 | 146 | 328.5 | |
| L-Histidine-HCl—H2O | 0.16245 | 210 | 34.1145 | |
| L-Isoleucine | 0.5125 | 131 | 67.1375 | |
| L-Leucine | 0.52075 | 131 | 68.21825 | |
| L-Lysine | 0.5735 | 183 | 104.9505 | |
| hydrochloride | | | | |
| L-Methionine | 0.137 | 149 | 20.413 | |
| L-Phenylalanine | 0.26125 | 165 | 43.10625 | |
| L-Proline | 0.1125 | 115 | 12.9375 | |
| L-Serine | 0.2875 | 105 | 30.1875 | |
| L-Threonine | 0.17625 | 119 | 20.97375 | 42.39375 |
| L-Tryptophan | 0.05255 | 204 | 10.7202 | |
| L-Tyrosine-2Na—2H2O | 0.26 | 225 | 58.5 | |
| L-Valine | 0.5395 | 117 | 63.1215 | |
| Linoleic acid | 0.000075 | 280 | 0.021 | |
| Lipoic acid | 0.00037025 | 206 | 0.076272 | 0.078795 |
| Magnesium chloride | 0.225 | 95 | 21.375 | |
| Magnesium sulfate | 0.50825 | 120 | 60.99 | |
| Niacinamide | 0.0206 | 122 | 2.5132 | |
| Phenol red | 0.01805 | 398 | 7.1839 | 7.5023 |
| Potassium chloride | 4.435 | 75 | 332.625 | |
| Putrescine-2HCl | 0.0512575 | 161 | 8.070246 | |
| Pyridoxine | 0.0123 | 206 | 2.5338 | 1.611195 |
| hydrochloride | | | | |
| Pyruvic acid | 0.25 | | 0 | 27.5 |
| Riboflavin | 0.00070525 | 376 | 0.265174 | 1.9796 |
| Sodium chloride | 118.085 | 58 | 6848.93 | 6846.465 |
| Sodium bicarbonate | 36.375 | 84 | 3055.5 | 2935 |
| Sodium phosphate, | 0.33975 | | 0 | 78.142 |
| mono. | | | | |
| Sodium phosphate, | 0.125 | | | 53.256 |
| dibas. | | | | |
| Sodium pyruvate | 1.125 | 110 | 123.75 | 96.25 |
| Thiamine | 0.00775 | 337 | 2.61175 | |
| hydrochloride | | | | |
| Thymidine | 0.001125 | 242 | 0.27225 | |
| Vitamin B12 | 0.000625 | 1355 | 0.846875 | 0.508125 |
| Zinc sulfate | 0.001125 | 288 | 0.324 | |
| Creatine | 3.355 | | 500 | |
| B-27+ Retinoic acid | 0.038194444 | | 1 ml/L | |
| bFGF | | | | 10 ng/ml |
| EGF | | | | 5 ng/ml |
| Kanamycin | | | | 1 ml/L |
| Insulin | | | 5 mg/L | 7.5 mg/L |
| Sodium Selenite** | | | 4 mg/L | |
| Transferrin | | | 25 mg/L | |
| Pen/Strep | | | | 0.750 ml/L |
| Normocin*** | | | | 1 ml/L |

*This column shows altered concentrations (in mg/L only) for certain components that are used in combination with the other components (at the disclosed concentrations) for particular embodiments.
**Sodium Selenite is included in particular embodiments.
***Normocin is included in particular embodiments.

In certain embodiments, the invention provides a culture medium for pluripotent stem cells that induces the specification of embryoid bodies formed therefrom to the oligodendroglial phenotype. Pluripotent/mutipotent stem cells, e.g., embryonic stem cells (ES) and/or induced pluripotent stem (iPS) cells, can be specified into oligodendrocytes by using a method for producing oligodendroglial cells from rodent, non-human primate, and/or human embryonic stem cells (e.g., hES) and/or induced pluripotent stem cells (e.g., hiPS), starting with the treatment of embryoid bodies (EBs) in suspension in non-adherent substrate with the culture medium described in table 5.

TABLE 5
Medium for Specification of Multipotent (iPS/ES) Stem Cells to
Oligodendrocyte Progenitors (iP/ESS-OLM)

| | Molarity | Formula | |
|---|---|---|---|
| Components | (mM) | Weight | mg/L |
| Biotin | 0.000011075 | 244 | 0.002702 |
| Calcium chloride | 1.14975 | 111 | 127.6223 |
| Choline chloride | 0.0552 | 140 | 7.728 |
| Cupric sulfate | 0.0000038 | 250 | 0.00095 |
| D-Calcium | 0.00555 | 477 | 2.64735 |
| pantothenate | | | |
| D-Glucose | 17 | 180 | 3060 |
| D-Galactose | | | 4600 g/L |
| Ethanolamine | 0.125 | 61 | 7.625 |
| Ferric nitrate | 0.000154 | 404 | 0.062216 |
| Ferrous sulfate | 0.001125 | 278 | 0.31275 |
| Folic acid | 0.00689 | 441 | 3.03849 |
| Fumaric acid | 0.25 | | 0 |
| Glycine | 0.26835 | 75 | 20.12625 |
| Hepes | 3.75 | | 0 |
| Hydroxybutyrate | 0.25 | | 0 |
| Hypoxanthine-Na | 0.01125 | | 2.40 |
| i-Inositol | 0.0625 | 180 | 11.25 |
| L-Alanine | 0.0375 | 89 | 3.3375 |
| L-Arginine | 0.624 | 211 | 131.664 |
| hydrochloride | | | |
| L-Asparagine-H2O | 0.0375 | 150 | 5.625 |
| L-Aspartic acid | 0.025 | 133 | 3.325 |
| L-Cysteine-HCl—H2O | 0.075 | 176 | 13.2 |
| L-Cystine-2HCl | 0.125 | 313 | 39.125 |
| L-Glutamic Acid | 0.025 | 147 | 3.675 |
| L-Glutamine | 2.25 | 146 | 328.5 |
| L-Histidine-HCl—H2O | 0.16245 | 210 | 34.1145 |
| L-Isoleucine | 0.5125 | 131 | 67.1375 |
| L-Leucine | 0.52075 | 131 | 68.21825 |
| L-Lysine | 0.5735 | 183 | 104.9505 |
| hydrochloride | | | |
| L-Methionine | 0.137 | 149 | 20.413 |
| L-Phenylalanine | 0.26125 | 165 | 43.10625 |
| L-Proline | 0.1125 | 115 | 12.9375 |
| L-Serine | 0.2875 | 105 | 30.1875 |
| L-Threonine | 0.17625 | 119 | 20.97375 |
| L-Tryptophan | 0.05255 | 204 | 10.7202 |
| L-Tyrosine-2Na—2H2O | 0.26 | 225 | 58.5 |
| L-Valine | 0.5395 | 117 | 63.1215 |
| Linoleic acid | 0.000075 | 280 | 0.021 |
| Lipoic acid | 0.00037025 | 206 | 0.076272 |
| Magnesium chloride | 0.225 | 95 | 21.375 |
| Magnesium sulfate | 0.50825 | 120 | 60.99 |
| Niacinamide | 0.0206 | 122 | 2.5132 |
| Phenol red | 0.01805 | 398 | 7.1839 |
| Potassium chloride | 4.435 | 75 | 332.625 |
| Putrescine-2HCl | 0.00037575 | 161 | 17.26 |
| Pyridoxine | 0.0123 | 206 | 2.5338 |
| hydrochloride | | | |
| Progesterone | 25 nm | | |
| Pyruvic acid | 0.25 | | 0 |
| Riboflavin | 0.00070525 | 376 | 0.265174 |
| Sodium chloride | 118.085 | 58 | 6848.93 |
| Sodium bicarbonate | 36.375 | 84 | 5870 |
| Sodium phosphate, | 0.33975 | | 71 |
| mono. | | | |
| Sodium phosphate, | 0.125 | | 53.25 |
| dibas. | | | |
| Sodium pyruvate | 1.125 | 110 | 123.75 |
| Sodium selenite | | | 8 |
| Thiamine | 0.00775 | 337 | 2.61175 |
| hydrochloride | | | |
| Thymidine | 0.001125 | 242 | 0.27225 |
| Vitamin B12 | 0.000625 | 1355 | 0.846875 |
| Zinc sulfate | 0.001125 | 288 | 0.324 |
| Creatine | 3.355 | | 25/100 ml |
| B-27 | 0.038194444 | | 2 ml/L |
| bFGF | | | 0 |
| EGF | | | 0 |
| Kanamycin | | | 1 ml/L |
| Insulin | | | 10 mg/L |
| Transferrin | | | 150 mg/L |
| Pen/Strep | | | 0.5 ml/L |
| Normocin | | | 1 ml/L |

The invention also includes embodiments in which one or more of the components in Tables 3, 4, or 5 is employed at a concentration that differs from that shown by 0.5%, 1.0%, 1.5%, 2%, 2.5%, 3.0%, 3.5%, 4.0%, 4.5%, or 0.5% or by a percentage falling within a range having any of these values as endpoints (e.g., a percentage falling within the range of 1.0%-2.5%, e.g., 2.3%). The concentration of the component may be higher or lower than that shown in Tables 3 or 4.

### Cell Culture Methods

The Stem Cell Medium described above can be used to propagate and maintain neural stem cells (NSC), as described in Espinosa-Jeffrey et al., “Selective Specification of CNS Stem Cells Into Oligodendroglial or Neuronal Cell Lineage: Cell Culture and Transplant Studies,” J. Neuroscience Res. 69:810-25 (2002) (which is hereby incorporated by reference in its entirety and specifically for its description of methods and compositions for generating neural stem cells and OL), and Espinosa-Jeffrey et al., “Culture System for Rodent and Human Oligodendrocyte Specification, Lineage Progression, and Maturation,” Curr. Protocols in Stem Cell Biol. 2D.4.1-2D.4.26 (2009) (which is hereby incorporated by reference in its entirety and specifically for its description of methods and compositions for generating neural stem cells and OL and is described below as Example 1). In particular embodiments, the Stem Cell Medium described above can be used in essentially the same manner as STM-IIc of Example 1.

The Oligodendrocyte Specification Medium (OSM) can be used to induce the specification of NSC to the oligodendrocyte (OL) phenotype, also as described in these references. The OSM described above can be used in essentially the same manner as OSM-II of Example 1. Furthermore, the two novel media formulas described herein can be used in combination with previously described culture media for OL maturation, “GDM” and “OLDEM” (a nutrient formula that supports OL complete development to reach their functional myelinating stage), to generate mature oligodendrocytes.

The OSM described above is designed specifically to use two sources of iron and two sources for energy metabolism. Both STM-IIc and OSM-II may, in certain embodiments, favor the growth of some astrocytes over time in these cultures, as compared to the Stem Cell Medium and OSM described above. Since reports in the literature have described methods that yield oligodendrocytes in 2 months to 6 months, astrocyte numbers would increase, producing a mixed glial culture. A culture that uses the Stem Cell Medium and OSM described above, rather than STM-IIc and OSM-II would have virtually no astrocytes, which is desirable if the goal is to design cell replacement therapies to recover neural function.

The Medium for Specification of Multipotent (iPS/ES) Stem Cells to Oligodendrocyte Progenitors (iP/ESS-OLM) can be used for induced pluripotent stem cells to specify embryoid bodies to the oligodendroglial phenotype. This medium has been designed to be used in conjunction with laminin or matrigel substrata, while working with human cells; other mammalian cells can be cultured in this manner or on poly-ornithyne or poly-L-Lysine. Combinations of antibodies against oligodendrocyte surface markers can also be used as substrata in the short term to select for the desired subpopulations, i.e. early OL progenitors that are selectable with the A2B5 antibody or more mature cells with O4 and other antibodies for oligodendrocyte surface markers.

This modality can also be used to maintain the attached cells at a specific developmental stage within the OL lineage as shown in example #1. This also applies to undifferentiated neural stem cells that can be maintained at this stage in a sustained manner when seeded on anti-PSA-NCAM antibody (Espinosa et la., 2002, 2009).

The sustainability of the OLP phenotype, expansion, long-term maintenance, and manipulation for their study and for further transplantation in cell-based therapies is affected by the use of the adequate substrate and the specific cell culture medium, which should be replenished to sustain the stoichiometry of the complete cell culture medium in a milieu that mimics the in vivo cell niche when culturing these cells. The use of a variety of specific cocktails, simultaneously or in tandem, depending of the cell line and/or source, also the desired results. The enrichment of oligodendrocyte populations requires the use of inhibitors for mesoderm and endoderm. Differences have been found in commitment/differentiation potentials among human pluripotent cell lines and therefore these culture media can be adjusted, depending on the particular cell line/type being used, to provide the desired results. In certain embodiments, an initial treatment can be carried out for a period of 1 to 7 days with Rock inhibitor, dorsomorphin and SB431542, as described by Kim, DS., et al. Robust enhancement of neural differentiation from human ES and iPS cells regardless of their innate difference in differentiation propensity. Stem Cell Rep 6:270-281 (2010) (incorporated by reference in its entirety for this description).

The main advantage over any other media described in the literature to generate oligodendrocytes from ES cells or from hiPS is that with the iP/ESSOLM (shown in Table 5), oligodendrocytes progenitors appear faster.

Furthermore, with iP/ESSOLM, the numbers of cells undergoing specification to the OL phenotype can be increased by the use of sonic hedgehog (SHH) and retinoic acid (RA) as described by many authors, including Hu B Y, Du Z W, Li X J, Ayala M, Zhang S C. Human oligodendrocytes from embryonic stem cells: conserved SHH signaling networks and divergent FGF effects. Development. 2009 May; 136(9): 1443-52. SourceDepartment of Anatomy and Neurology, University of Wisconsin-Madison, Madison, Wis. 53705, USA (incorporated by reference in its entirety for this description). See also, Stuart M. Chambers, Christopher A. Fasano, Eirini P. Papapetrou, Mark Tomishima, Michel Sadelain, and Lorenz Studer. Highly efficient neural conversion of human ES and iPS cells by dual inhibition of SMAD signaling. Nat Biotechnol. 2009 March; 27(3): 275-280 for further methods that can be used in conjunction with the iP/ESS-OLM (this reference is incorporated by reference in its entirety for this description).

### Example 1

### Culture System for Rodent and Human Oligodendrocyte Specification, Lineage Progression, and Maturation

### Abstract

Here we document protocols for the production, isolation, and maintenance of the oligodendrocyte phenotype from rodent and human neural stem cells. Our unique method relies on a series of chemically defined media, specifically designed and carefully characterized for each developmental stage of oligodendrocytes as they advance from oligo-dendrocyte progenitors to mature, myelinating oligodendrocytes.

### Introduction

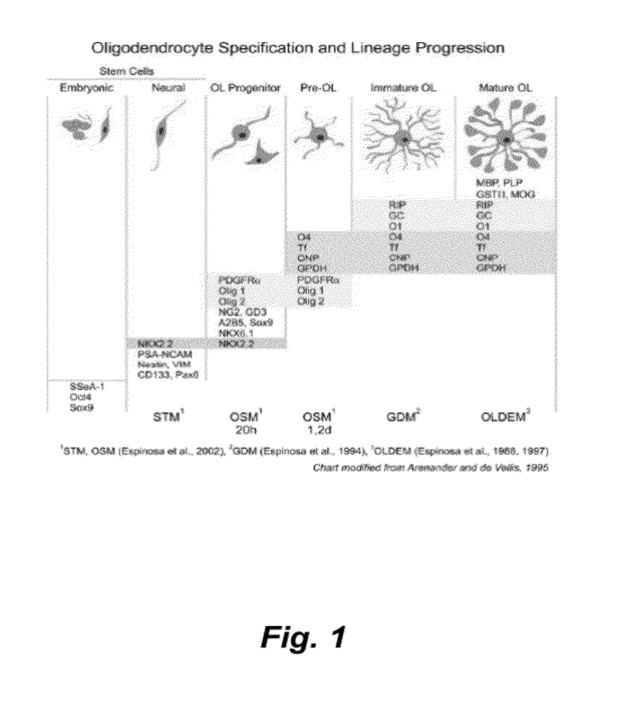

In this example, protocols are provided for the derivation, expansion, and maintenance of the oligodendrocyte (OL) phenotype from both rodent and human neural stem cells (NSC). This unique method utilizes chemically defined media, each formulated and carefully characterized for specific developmental stages of OL as they advance from OL progenitors (OLP) to mature myelinating OL (FIG. 1). By providing hNSC with the nutrients specifically required at a particular moment in OL development, our system allows for the propagation of OL at a desired stage from OLP to mature premyelinating OL. Therefore, lineage progression can be manipulated by controlling the duration of a given developmental stage as needed, in a more “natural” manner, and without using gene transfer (Park et al., 2002b; Kim, 2004; Muller et al., 2006; Ahn et al., 2008), cocultures, or undefined substrates such as cell line—derived conditioned medium or animal serum.

### Preparation of Embryonic Neural Stem Cells (NSC)

The methodology described in this example can be used to isolate and derive NSC lines from various species. Specific methods for the derivation of human NSC are detailed elsewhere (Svendsen et al., 1999; Villa et al., 2000; Palmer et al., 2001; Schwartz et al., 2003; Kim et al., 2006; De Filippis et al., 2007; Kim et al., 2008; Wakeman et al., 2009).

### Isolation of Rodent Neural Stem Cells—Basic Protocol I

In this protocol, 1 to 14 embryos can produce a successful preparation because stem cells can be propagated many times to obtain the desired yield.

Materials

One timed-pregnant, embryonic day 14 to 16 (ED14 to ED16) Sprague-Dawley rat (Charles River Laboratories)

Basal stem cell medium (STM-II; see recipe) supplemented with 1% (w/v) BSA (Sigma, cat no. A-3156

Phosphate-buffered saline (PBS; Sigma, cat. no. P-5368)

Complete stem cell medium (STMIIc; see recipe)

Dissection instruments, sterile: Mayo scissors (Fine Science Tools, cat. no. 14010-17), Lister scissors (Fine Science Tools, cat. no. 14131-14), blunt-pointed forceps (Fisher, cat. no. 08-887), iris scissors (Fine Science Tools, cat. no. 14060-09), Maria iris forceps (Fine Science Tools, cat. no. 11373-12), Dumont #7 forceps (Fine Science Tools, cat. no. 11297-10), 140-μm and 230-μm sieves (Cellector, E-C Apparatus Corp.; www.thermo.com), 20-ml syringe (Kendall, cat. no. 520673; www.kendallhq.com), 18-G Quincke spinal luer-lock needle for dissociation (100-mm length; Unimed; www.unimed.ch/)

100×15-mm petri dish (bacterial grade, non TC-treated; BD Falcon, cat. no. 351029)

50-ml and 15-ml conical tubes Centrifuge (e.g., IEC Clinical) 100-mm anti-PSA-NCAM coated dishes (Support Protocol 1)

37° C., 4.5% CO₂ incubator (adjustable to 5% if growth is slow), 95% humidity

Additional reagents and equipment for isoflurane anesthesia of the mouse, assessing cell viability (Support Protocol 2), and counting cells using a hemacytometer

NOTE: All dissection instruments, plasticware, and glassware must be sterile.

Collect the Embryo Brains

1. Prepare the work area and sterile tools in a biosafety hood.

2. Euthanize the rat by isoflurane inhalation

3. Dissect and remove the uterus. Collect the placenta-containing embryos and place in basal stem cell medium containing 1% BSA at room temperature in a non-tissue-culture-treated 100-mm-diameter petri dish.

4. Remove the embryos from their placenta. Place them in a 100-mm petri dish containing PBS at room temperature. Remove the cerebellum from each embryo.

5. Dissect out the brain of each embryo and place in STM-II complete (STMIIc) medium at room temperature in a 100-mm non-tissue-culture-treated petri dish.

6. Separate the cortex from the rest of the brain and remove the meninges with forceps. Once devoid of meninges, combine cortex and pons (i.e., the rest of the brain).

Isolate the Cells

7. Combine the tissue of all the brains without meninges in a 15-ml conical tube. Mechanically dissociate with the 18-G needle (attached to a 20-ml syringe) by gently aspirating the brain pieces (10 times) and releasing the suspension slowly with the needle against to the wall of the tube (try to minimize foaming).

8. Centrifuge 8 min at 450×g, room temperature. Recover the supernatant with the cells in suspension and transfer to a 15-ml tube.

9. Add 2 to 4 ml of STMIIc to the chunks left over in the dissociation tube and dissociate again five to eight times.

In place of steps 7 to 9, you can dissociate the cells for 2.5 min in a Stomacher 80 (Seward; www.brinkmann.com).

10. Filter the suspension of dissociated cells first through the 230-μm sieve and then through the 140-μm sieve to remove cell clusters.

11. Rinse the two sieves sequentially with 2 ml STM-II medium containing 1% BSA at room temperature, and add this medium to the tube containing the cells.

12. Collect the cells by centrifugation 8 min at 450×g, room temperature.

13. Gently discard the supernatant.

14. Resuspend the cell pellet in 4 ml of complete stem cell medium (freshly prepared), and gently dissociate the pellet with the syringe and needle by aspirating it up and down twice.

Initiate the Cultures

15. Assess cell viability (Support Protocol 1), count cells using hemacytometer, and plate onto fresh PSA/NCAM-coated dishes (2×10⁶ cells/100-mm dish in 7 ml of STMIIc medium).

16. Incubate plated cells overnight at 37° C. with 4.5% CO₂ and 95.5% humidity.

Younger cells do not yet express PSA-NCAM and will remain floating as small clusters, whereas the older cells will attach overnight.

Collect Conditioned Medium (CM)

17. On the next day, transfer the nonattached cells to a 15-ml conical tube, pellet cells by centrifugation for 8 min at 500×g, room temperature, and remove and save the conditioned medium (CM).

18. Collect, filter, and save the conditioned medium at 4° C. for immediate use (or frozen for later use).

CM is an excellent supplement to start NSC cultures from frozen stocks.

19. Allow attached cells (see step 16) to grow to 70% to 90% confluency.

20. Resuspend the pellet from step 17 and dissociate in 4 ml of fresh STMIIc medium by passing through a 14-G needle eight times. Bring the volume to 8 ml with conditioned medium and plate on additional anti-PSA-NCAM coated plates.

Alternatively, to dissociate the cells, place 1.0 ml of the cell suspension in a sterile 75-ml Erlenmeyer flask in 25 ml of STMIIc, and incubate with shaking at 37° C.

21. Feed all cells every other day by removing ⅓ of the culture medium and adding the same volume of fresh STMIIc.

22. Switch the cells from 4.5% to 5.0% CO₂ only if the cells are growing slowly. Leave them at 4.5% if the color of the medium stays red/orange.

23. Optional: To assess the phenotype of these cells, perform immunocytochemistry (Espinosa et al., 2002).

### Preparation of Anti-PSA-NCAM-Coated Dishes for Selecting NSC by Immunopanning—Support Protocol 1

The following method was developed based on published work (Wysocki and Sato, 1978; Williams and Gard, 1997) to isolate the rodent NSC population from the other cell populations in the brain during initial plating. We also use anti-PSA-NCAM coated dishes to propagate rodent NSC in two-dimensional cultures (Espinosa-Jeffrey et al., 2002). Please refer to the literature for specific methods on the selection of human NSC during primary derivation (Wakeman et al., 2009).

We have chosen immunopanning as opposed to flow cytometric cell sorting because we find that cell survival approaches 100% when selecting the desired cell type via immunopanning We know from both the experience of other scientists and our own that the survival rates are never this high when using FACS. Moreover, immunopanning can be performed in the standard culture vessel and is as simple as plating the cells on the adequate substrate for cell selection.

Materials

50 mM Tris.Cl, pH 9.5

Bovine serum albumin (BSA; Sigma, cat. no. A-3156)

Anti-PSA-NCAM antibody (Iowa DSHB, http://dshb.biology.uiowa.edu/, cat. no. 5A5)

Phosphate-buffered saline (PBS; Sigma, cat. no. P-5368)

100×15-mm petri dish (bacterial grade, non-TC-treated; BD Falcon, cat. no. 351029)

1. Prepare the immunopanning cocktail:
50 mM Tris.Cl, pH 9.5 containing: 1% (w/v) BSA
50 μg/ml anti-PSA-NCAM antibody.

2. Coat the bottom surface of 100-mm non-tissue-culture-grade petri dishes by adding 4 to 5 ml per dish of the anti-immunopanning cocktail and incubating 30 min at 37° C.

Flasks may also be coated by this protocol: use 2.5 ml to coat a 12.5-cm² flask or 5 ml to coat a 75-cm² flask.

3. Remove the cocktail. Wash petri dishes three times, each time with 5 ml PBS, then once with 5 ml PBS containing 1% BSA just before using. Do not allow the plates to dry.

4. Cover extra plates (still containing the PBS/BSA) with foil and store at 4° C. for up to 10 days.

### Assessing Cell Viability—Support Protocol 2

Cell viability can be determined with the SYTOX blue nucleic acid stain (Molecular Probes/Invitrogen). Cells with compromised plasma membranes are labeled by SYTOX binding to nucleic acids and detected by fluorescence microscopy.

Materials

Tris-buffered saline (TBS; see recipe)

Phosphate-buffered saline (PBS; Sigma, cat. no. P-5368)

1 μM SYTOX blue nucleic acid stain (Invitrogen Molecular Probes, cat. no. S7020) in PBS

Microscope slides and coverslips

Fluorescence microscope

1. Gently wash cells three times, each time with 5 ml TBS.

2. Harvest cells with a cell scraper and transfer them to a 15-ml tube. Resuspend in 2 ml PBS. Do not use enzymes.

3. Add 1 μl of 1 μM SYTOX to each tube (final concentration, 5 nM).

4. Incubate 12 min.

5. Remove the solution and wash the cells five times, each time with 5 ml TBS, centrifuging 3 min at 500×g, room temperature, each time.

6. Resuspend the cells in 1 ml/tube of PBS. Take an aliquot and add a drop to a microscope slide. Add a coverslip and examine with a fluorescence microscope.

7. Determine the number of positive cells per random field in a fluorescence microscope and record as a percentage of the total number of cells in the field.

### Propagation of Rodent NSCS as Two-Dimensional Cultures—Basic Protocol 2

NSCs can be propagated in two-dimensional (2-D) or three-dimensional (3-D) cultures. When attached, NSCs (2-D cultures) tend to grow faster and are therefore ideal for creating a large cell stock quickly before starting specific studies. In addition, we have developed a new method for expansion and maintenance of human NSC in NB-B-27 medium (see Reagents and Solutions) as multilayer adherent network (MAN) cultures, with increased proliferation rates compared to standard sphere-forming assays. In order to accommodate the difference in basal media, human NSC can either be initially derived in STMIIc media (replacing NB-B-27), or previously established hNSC cultures may be slowly transitioned away from the basal NB-B-27. Simply substitute 25% STMIIc for 1 week, followed by successive weeks at 50%, 75%, and finally 100% STMIIc after 1 month.

This procedure can be used every time cells are replated.

Materials

Cultures of freshly isolated neural stem cells (Basic Protocol 1) or their progenitors Hanks' buffered salt solution (HBSS) without Ca²⁺ or Mg²⁺

Complete stem cell medium (STMIIc; see recipe)

Cell Freezing Medium, serum-free, 1×(Sigma, cat. no. C2639)

Cell scraper

15-ml conical tubes

Centrifuge (e.g., IEC Clinical)

20-ml syringe

18-G Quincke spinal luer-lock needle for dissociation (100-mm length; Unimed; www.unimed.ch/)

12.5-cm² and 75-cm² tissue culture flasks (Falcon), anti-PSA-NCAM-coated (Support Protocol 1)

1.2-ml cryovials

Additional reagents and equipment for counting cells using a hemacytometer and freezing cells (Support Protocol 3)

NOTE: All steps are performed at room temperature (20° C.).

Collect the Cells

1. When confluency of the neural stem cells has been reached, remove the supernatant conditioned medium (CM; save for step 4), add 5 ml of HBSS without Ca²⁺ or Mg²⁺, detach the cells with a cell scraper, and transfer to a 15-ml conical tube (accommodating cells from one to three dishes).

2. Rinse the dish once with 2 ml HBSS (without Ca²⁺ or Mg²⁺), add it to the tube, and centrifuge 8 min at 450×g. Discard the supernatant.

3. Resuspend the cell pellet in 3 ml STMIIc, dissociate gently using 18-G needle and syringe, and centrifuge 5 min at 450×g.

4. Resuspend cells in 2 ml of a freshly prepared mixture of 2 parts STMIIc and 1 part CM (use CM from step 1). Replate cells on anti-PSA-NCAM-coated plates as described in Basic Protocol 1.

If you have repeated this process several times and the cell pellet is 0.5 ml volume or larger, divide the cell suspension into two parts. One part of the suspension will be used to start a frozen stock (see Support Protocol 3). The second half of the cell suspension is further dissociated using a needle (as described in Basic Protocol 1; however, the sieves (used in step 10 of Basic Protocol 1) are not necessary (single-cell suspension is obtained using needle and syringe), and replated as described below.

Replate the Cells

5. Count the number of cells/ml and adjust the volume to 15 ml with a freshly prepared mixture of 2 parts STMIIc and 1 part CM (use CM from step 1).

6. Plate 2 ml of the cell suspension in each of five 12.5-cm² cell culture flasks coated with anti-PSA-NCAM.

Passage the Cells

7. Feed cells with a freshly prepared mixture of 2 parts STMIIc and 1 part CM every other day until they reach 80% to 90% confluency, and repeat steps 1 to 7 to increase the number of cells.

8. When four or more 12.5-cm² flasks reach confluency, harvest the cells as described above, and seed the equivalent content of cells from three 12.5-cm² flasks into one 75-cm² flask coated with anti-PSA-NCAM Feed the cells with a freshly prepared mixture of 2 parts STMIIc and 1 part CM in a total volume of 10 ml/flask.

9. After 1 to 2 days, check to see if the culture medium is red. If the medium is turning orange, add 3 ml of STMIIc. Repeat this step as needed. Add 3 ml of STMIIc every day only if the medium changes color.

If cells seem not to grow, but look healthy, or if the culture medium is not red but purple, you will need to remove ½ of the plating medium and bring the volume up to 10 ml with a freshly prepared mixture of 2 parts STMIIc and 1 part CM.

If the opposite is true and the culture medium turns orange overnight, the cells have proliferated heavily, and you will need to replace the culture medium and seed more 75-cm² flasks (one 75-cm² flask per three 12.5-cm² flasks).

10. Optional: When cells reach confluency, freeze the contents of one 75-cm² flask (Support Protocol 3) in 1 ml of freezing medium in a cryovial.

When propagating cells to create frozen stocks, we strongly recommend maintaining a “mother flask” by scraping most, but not all of the cells attached to the flask. After removing the detached cells, feed the motherflask with a 1:1 mixture of fresh STMIIc and CM to ensure continuity of these cultures (in case replated cells do not look healthy, grow slowly, or die).

11. Optional: After accumulating at least 10 to 15 vials of cryopreserved NSC in a frozen stock, grow NSCs as neurospheres (three-dimensional) for slower growth, allowing more time to devote to experiments.

While cells are floating, even in STMIIc their metabolism seems slower, but if replated they behave normally.

### Formation, Propagation, and Maintenance of Neurospheres in Three-Dimensional Cultures—Alternate Protocol

Suspension aggregate, or “neurosphere,” three-dimensional cultures are an alternative strategy to propagate NSCs at a slower pace than that of attached cells, while preserving most of the standard characteristics of a proper NSC. NSC suspension cultures are started from freshly dissociated NSC two-dimensional cultures (Basic Protocol 2) and grown in sterile Erlenmeyer flasks to prevent attachment and encourage free-floating spherical growth.

Materials

Conditioned medium (see Basic Protocol 2) Complete stem cell medium (STMIIc; see recipe) Established NSC cultures (Basic Protocol 2)

Glass Erlenmeyer flasks, 25-ml or 50-ml with cap

37° C. incubator with shaker

20-ml syringe

18-G Quincke spinal luer-lock needle for dissociation (100-mm length;

Unimed; www.unimed.ch/)

50-ml conical tubes

Centrifuge (e.g., IEC Clinical)

0.22-μm sterile filters

NOTE: All steps are performed at room temperature (20° C.).

1. After harvesting cells as described in Basic Protocol 2, resuspend the cells from one 75-cm² flask in 6 ml fresh STMIIc.

2. Place 15 ml of fresh STMIIc and 3 ml of conditioned medium (CM) into a 25- or 50-ml Erlenmeyer flask (depending on number of cells).

3. Add 2 ml of the cell suspension (freshly harvested from two-dimensional cultures as described in Basic Protocol 2), and close the top of the flask partially to allow for O₂/CO₂ exchange.

Thus, one 75-cm² flask will result in three Erlenmeyer flasks of neurospheres. Place the flask, continuously shaking at 90 rpm, in the incubator.

If placing a shaker in the incubator is not an option due to safety regulations, the cell suspension may be placed directly into two noncoated petri dishes (bacterial grade) to prevent cell attachment.

4. Add 1.5 ml fresh STMIIc, every other day, and dissociate routinely (three times gently) with a syringe and needle in the same flask (as described for pellet dissociation in Basic Protocol 2) to keep the spheres at a small size.

This process allows for increased sphere formation without the negative potential for spontaneous differentiation. It also allows cells more exposure to the fresh nutrients in the culture medium, helping preserve “sternness” in all cells.

5. When the culture medium turns orange overnight, collect the contents of the Erlemeyer flask with a pipet and place into one 50-ml conical tube. Centrifuge for 6 min at 450×g.

6. Slowly collect the CM (supernatant), filter (0.22-μm), and save for replating cells.

7. Resuspend the pellet in 4 ml of STMIIc to dissociate larger spheres. Add 21 ml of a freshly prepared mixture of 2 parts STMIIc and 1 part CM.

At this point, the neurospheres should all be easily dissociated.

If some spheres remain large in spite of repeated dissociation, use the sieves (see Basic Protocol 1 materials list) to eliminate the large clumps, instead of vigorously dissociating them. This step will prevent significant cell death at the time of replating.

8. Seed cells on desired containers for experiments, or continue to propagate NSCs as two- or three-dimensional cultures (see Basic Protocol 2).

### Cryopreservation/Thawing of NSC Stocks—Support Protocol 3

We recommend collecting cells for frozen stocks at low passage number. Human NSC are cryopreserved using modified methods found elsewhere (Wakeman et al., 2009). In addition, the method formerly described for rat and mouse NSC (Espinosa-Jeffrey et al., 2002) can also be used to stock human NSC.

We recommend the use of serum-free freezing medium as well as all other animal-free components as indicated within this example.

Materials

NSC cultures ready for freezing (Basic Protocol 2)

Hanks' balanced salt solution (HBSS) without Ca²⁺ or Mg²⁺ Complete stem cell medium (STMIIc; see recipe)

Cell Freezing Medium, serum-free, 1×(Sigma, cat. no. C2639) Liquid nitrogen

Conditioned medium (CM; see Basic Protocol 2)

Cell scrapers

Centrifuge (e.g., IEC Clinical)

20-ml syringe

18-G Quincke spinal luer-lock needle for dissociation (100-mm length; Unimed; www.unimed.ch/)

1.2-ml cryovials

Cryogenic slow-freezing chamber (Nalgene, cat. no. EW-44400-00) 2-ml tubes (Fisher)

Anti-PSA-NCAM coated tissue culture vessels (Support Protocol 1)

Additional reagents and equipment for testing cell viability (Support Protocol 2). NOTE: All steps are performed at room temperature (20° C.).

Collect and Freeze the NSC

1. Allow NSCs to grow to 70% to 90% confluency. Remove all of the cell culture medium, add 5 ml of HBSS (without Ca² or Mg²) to each 100-mm petri dish or 10 ml to each 75-cm² flask, and detach cells by gently scraping the culturing surface.

2. Centrifuge the cells 8 min at 450×g, and resuspend in 3 ml STMIIc medium.

3. Gently dissociate cells using an 18-G needle and syringe, centrifuge 8 min at 450×g, and discard the supernatant.

4. Gently resuspend the pellet from one 100-mm petri dish or 75-cm² flask in 1 ml of serum-free freezing medium.

5. Transfer the contents to a 1.2-ml cryovial, and place the vial(s) in a cryogenic freezer container overnight for slow freezing.

6. Next day, place the vials in liquid nitrogen for long-term storage.

Thaw NSCs

7. To reanimate NSCs, defrost cryovials quickly in a 37° C. water bath, and transfer the contents of the vial to a 2-ml tube containing 1 ml of a freshly prepared mixture of 2 parts STMIIc and 1 part CM at 37° C.

8. Centrifuge gently 5 to 7 min at 350×g.

9. Remove the supernatant, add 1 ml of a freshly prepared mixture of 2 parts STMIIc and 1 part CM at 37° C., resuspend the cell pellet, and remove a small aliquot to test the initial cell viability.

10. Count the number of viable cells in the tube (about 1×10⁶ cells expected) as described in Support Protocol 2.

11. Plate cells onto an anti-PSA-NCAM-coated vessels (petri dishes or 75-cm² flasks, plate the equivalent of 1 vial/75 cm² flask).

If the yield is lower, use 25-cm² flasks to increase the cell density necessary for healthy growth. Seeding low-density cultures in large containers decreases the proliferation rate and might be detrimental to the culture.

12. To propagate NSCs after replating, proceed as described in Basic Protocol 2.

Alternatively, when cells have reached 90% confluency, either freeze them or use them for experiments.

### Oligodendrocyte Commitment in Two- and Three-Dimensional Cultures—Basic Protocol 3

During development, the nutritional and environmental needs of cells change as they lose multipotency and become lineage restricted. The present system is based on the modification of nutrients contained in the cell culture medium and the percentage of CO₂ needed to optimize and direct lineage restriction towards the oligodendrocyte (OL) phenotype. Like NSCs, OL progenitors (OLPs) can be propagated in two- and three-dimensional cultures. When attached (two-dimensional cultures), OLPs grow faster and, thus, two-dimensional cultures are ideal to create an OLP cell stock quickly before starting specific in vitro cell culture or in vivo transplantation studies.

Materials

NSC cultures (2-D or 3-D; Basic Protocol 2 or Alternate Protocol) Hanks' balanced salt solution (HBSS) without Ca²⁺ or Mg²⁺

OL specification medium (OSM-II; see recipe)

Cell scraper

15-ml conical tubes

20-ml syringe

18-G Quincke spinal luer-lock needle for dissociation (100-mm length; Unimed; www.unimed.ch/)

Anti-IgM coated 100-mm petri dishes or tissue culture flasks: prepare as in Support Protocol 1 but substitute goat anti-IgM antibody (ABR, sold by Thermo Scientific, cat. no. PA1-86106) for anti-PSA-NCAM antibody

37° C., 4.5% CO₂ incubator

12-ml syringe (Tyco Healthcare, cat. no. 512852)

Additional reagents and equipment for maintaining cells (Basic Protocol 1). NOTE: All steps are performed at room temperature (20° C.).

NOTE: We recommended precalibrating the percentage of CO₂ 1 day before plating the cells. If the incubator is shared with other people or needed at 5% for NSC propagation and maintenance, we recommend using tissue culture flasks for 2-D cultures instead of petri dishes. Close the cap of the flask completely and then open it one-quarter of a turn before placing in the incubator at 5% CO₂. For propagation and maintenance of OL spheres, the Erlenmeyer flask should also be kept open just enough to ensure O₂/CO₂ exchange. When using 4.5% CO₂, loosen the caps of the flasks until half-way open.

1. When NSCs reach confluency, remove the supernatant (CM; save for use in subsequent steps), and add 5 ml of HBSS without Ca²⁺ or Mg²⁺.

2. Detach the cells with a cell scraper, transfer into a 15-ml tube (which accommodates one to three petri dishes), rinse once with 2 ml of HBSS (without Ca²⁺ or Mg²⁺), and centrifuge 8 min at 450×g.

3. Resuspend the cell pellet in 3 ml OSM-II medium and gently dissociate three times using a syringe and 18-G needle. Centrifuge 8 min at 450×g to pellet the cells.

For Two-Dimensional OL Cultures

4a. Resuspend the cells in a freshly prepared 1:1 mixture of OSM-II and CM. Seed cells on anti-IgM coated dishes or flasks.

By using anti-IgM unconjugated antibody for coating, not only PSA-NCAM-positive cells will attach to the plate or dish, but also the cells that begin to express A2B5⁺ gangliosides. The panning strategy can be used at later stages to select OL cells at a single developmental stage, e.g., pre-OL, which can be selected using anti-O4.

The CM used here and in the following step is self-conditioned STMIIc.

5a. Maintain the cells as described in Basic Protocol 1 but using a mixture of 2 parts OSM-II and 1 part CM. From this point on, maintain the CO₂ concentration in the incubator at 4.5%.

Cells switch from NSCs to OLP within 20 hr in contact with OSM, at which time the cells start to express transferrin (TO.

6a. Feed the cells with a mixture of 2 parts OSM-II and 1 part CM (i.e., self-conditioned OSM-II) every other day until they reach 80% to 90% confluency (3 to 5 days).

This process can be repeated several times to attain a large number of cells for freezing (if desired).

For Three-Dimensional OL Cultures

4b. Alternatively, to grow OL spheres to create/enrich a frozen stock of OLP, place the equivalent of 2 mm² of cells (pellet size after cells are dissociated and in suspension) in a 25-ml Erlenmeyer flask with 15 ml of a mixture of 2 parts (10 ml) OSM-II and 1 part (5 ml) CM (i.e., self-conditioned OSM-II).

If the pellet is 4 mm² (˜12-15×10⁶ OLP), use a 50-ml Erlenmeyer flask. Prepare the cell suspension and place in a total volume of 25 ml of a mixture of 2 parts OSM-II and 1 part CM (i.e., self-conditioned OSM).

5b. Feed OL spheres with fresh OSM-II every other day by adding 3 ml of freshly prepared OSM-II (no CM).

6b. When spheres start to become larger than 2 mm, gently dissociate by aspirating them one to two times in the same flask with the 18-G needle using a 12-ml syringe (sterile).

7. When the culture medium starts to turn orange, recover and centrifuge the spheres from one flask, and split cells into more Erlenmeyer flasks (1 to 4).

These may be used for experiments or cryopreserved as previously described (Support Protocol 3).

Culturing Oligodendrocytes for Lineage Progression and Maturation—Basic Protocol 4

The nutritional needs for a committed cell within the OL lineage differ considerably as the cells progress and mature to the next developmental stage. These cells need to start synthesizing enzymes and proteins related to myelination; therefore, the energy demand is enormous compared to their earlier stage where migration and proliferation are the basic functions. The culture medium “GDM” (glial defined medium) was first designed to maintain 04⁺, GC^{+/-}, CNP^{+/-} cells. Later, we realized that GDM also induced the transition of OLP to pre-OL (Espinosa de los Monteros et al., 1997). OL can be sustained at a given developmental stage by keeping them in one of the stage-specific culture media described here.

Not all cell types offer the possibility for studying commitment and full differentiation when grown in culture, and one of the best examples comes from glial biology. Astrocytes grown in artificial cell culture conditions have allowed us to understand many of their functions and interactions with neurons and oligodendrocytes and how they play an integral part in mediating disease pathology; however, to our knowledge, there is no definitive proof of a fully matured, terminally differentiated astrocyte that can be studied throughout its progression and maturation in cell culture. In contrast to astroglial biology, we appear now to have the necessary tools to terminally differentiate oligodendrocytes in vitro, which even produce large amounts of compact myelin-like membranes in the absence of axons.

Most cell culture methods for maintaining OL only allow the researcher to address commitment, survival, or maintenance in a cell type that will, by default, tend to progress from the progenitor stage into a more mature stage uncontrollably (i.e., beyond control by the researcher) when maintained in a fairly rich environment conducive to and promoting myelinogenic properties. However, in our model, the design of several culture media specific for multiple OL developmental stages provides us with the ability to control lineage progression of normal OL at multiple lineage transitions. In addition, these subtype-specific media allow us to determine potential deficiencies in diseased or stressed OL derived from transgenic/mutant animals and tissues donated by human subjects. Therefore, development of OL-lineage-specific media formulations allows us to further model disease mechanisms and determine how they affect OL at different stages of development. Furthermore, we can then use these data to design specialized culture media aimed at either further protecting the cell or preventing specific mechanisms from potentially occurring in OL-related diseases, or as part of their inherent injury response. Utilizing this platform, we can apply high-throughput small-molecule libraries to our defined media and determine how specialized formulations may effectively aid in the restoration and repair of degenerating tissue.

Materials

OLPs (Basic Protocol 3, step 6a)

OL specification medium (OSM-II; see recipe)

GDM medium (see recipe)

Recombinant human basic fibroblast growth factor (bFGF; Invitrogen) OLDEM medium (see recipe)

Poly-D-lysine-coated wells/plates (see recipe)

Additional reagents and equipment for oligodendrocyte differentiation in two-dimensional culture (Basic Protocol 3)

Culture for Pre-OLs

1. In order to obtain pre-OL (along the OL lineage), plate OLPs using OSM-II (as in Basic Protocol 3).

As in previous steps, they may be propagated as OL spheres (three-dimensional) or as two-dimensional cultures on anti-IgM coated flasks or petri dishes, or directly on cell-culture grade plastic.

2. On the next day, remove one-half of the volume of the plating medium (OSM-II) and add the same volume of GDM. Continue incubation for a minimum of 2 days, or until 90% confluence is reached.

3. To obtain more OLP/pre-OL, grow cells as two- or three-dimensional cultures in the presence of bFGF. To keep progeny cells at the same stage as the parent cells, add 2 ml fresh GDM containing 20 ng/ml bFGF every other day until 90% confluence is reached.

For cell replacement therapies, we suggest using cells at this stage (1 to 2 days after plating without bFGF), as cells are still highly motile and readily migrate within the host post-natal and/or adult rodent brain and/or spinal cord.

4. To enhance maturation of cells into the next developmental stage, culture OL as two-dimensional cultures (Basic Protocol 3). Plate 110⁵ cells/ml in GDM for at least 2 days (if plated in GDM without bFGF), or 4 days (if plated in GDM with bFGF) without further bFGF supplementation.

After exposure to GDM, cells express myelin enzymes and proteins, and they display multipolar, branched cell processes, but not a myelin-like membrane. In addition, OL maintained in GDM for at least 4 days (without bFGF or any other factors) can be further induced to a fully mature myelinating stage.

5. To obtain fully mature OL, plate as two-dimensional cultures (Basic Protocol 3) at 1×10⁵ cells/ml onto poly-D-lysine coated wells/plates or uncoated petri dishes in 10 ml of a 1:1 mixture of GDM and OLDEM (OL maturation medium). Culture for 1 to 5 days, then replace with 100% OLDEM for further culture.

6. Every 4 days, feed the cells by replacing all of the culture medium with fresh OLDEM.

Myelinating OL express myelin markers.

The medium should look red, not orange. If it turns orange, add more medium while feeding the cells.

These cells will express myelin enzyme levels comparable to those found in pure myelin within 5 days after having been introduced to 100% OLDEM. As they mature, cells will synthesize myelin-like membranes in vitro even in the absence of neurons. They can be maintained for a number of weeks if they are kept subconfluent; however, if the culture becomes overcrowded, cells will deteriorate and die.

### Propagation of Oligodendrocytes for In Vitro Myelination Assays—Basic Protocol 5

To perform myelination studies in vitro, it is recommended to start with OL plated on plastic alone (rather than poly-D-lysine) and maintained in GDM for 2 days.

Materials

OL plated on (uncoated) plastic tissue culture dishes (from Basic Protocol 4, step 2)

GDM medium (see recipe)

Conditioned medium (from GDM; Basic Protocol 4)

OLDEM medium (see recipe)

Cell scraper

Neuronal cultures (Support Protocol 4) plated on poly-D-lysine-coated coverslips Complete Neurobasal-N medium for cortical neurons (see recipe)

40-pm cell strainers (BD Falcon, cat. no. 352340)

NOTE: All steps are performed at room temperature (20° C.).

1. Detach OL cells with cell scraper and centrifuge at 450×g, in the original culture medium.

2. Remove the supernatant and resuspend the cells in CM plus fresh OLDEM (1:2).

3. To prepare a single-cell suspension, which is necessary for the next step, remove any cell clusters by passing the cell suspension through a 40-μm sieve and wash the sieve as described in Basic Protocol 1, step 10.

4. Count the cells and adjust the cell suspension to ˜200,000 cells/ml in OLDEM medium.

5. At 10 days after plating, remove half the volume (250 μl) of culture medium from neuronal culture (Support Protocol 4) without disturbing the cells.

The neurons for coculture can be either cortical neurons or dorsal root ganglion cells.

6. Slowly add 200 μl of the OL suspension (from step 4) to the wells of one 24-well plate containing the neuronal cultures. To complete the original total volume in each well, add 50 μl/well of complete Neurobasal N medium for cortical neurons.

7. Follow the cocultures for at least 10 days. To feed, on day 5 after starting coculture, remove one half of the CM from each well (250 μl) and replace with fresh OLDEM.

Repeat OLDEM feeding once a week. There is no need to reapply Neurobasal N medium. If the cultures are not overcrowded, they can be kept for at least 4 weeks.

### Preparation of Cortical Neurons—Support Protocol 4

Cortical neurons are one cell type that is used for coculture with the OLs to assess myelination.

### Materials

Complete Neurobasal-N medium for cortical neurons (see recipe) Poly-lysine-coated (see recipe) coverslips in wells of 12- or 24-well plates 37° C. 4.5% CO₂ incubator, 95% humidified

Combustion Test Kit (Bacharach, cat. no. 10-500; www.bacharach-inc.com)

Additional reagents and materials for isolation of rodent brain cells (see Basic Protocol 1)

NOTE: All steps are performed at room temperature (20° C.).

1. Prepare and dissociate embryonic rat brain tissue as described in Basic Protocol 1, steps 1 to 8, except use complete Neurobasal-N medium in step 5 of that protocol (instead of STMIIc) and dissect the brains in Neurobasal-N medium.

2. Add 2 to 4 ml of Neurobasal-N medium to the chunks left over in the dissociation tube and dissociate again five to eight times.

3. Filter the suspension of dissociated cells through 230-pm and 140-pm sieves to remove cell clusters.

4. Rinse the two sieves sequentially with Neurobasal-N containing 1% BSA at room temperature as described in Basic Protocol 1, step 10, and add this medium to the tubes containing the cells.

5. Collect the cells by centrifugation in the culture tubes 8 min at 400×g.

6. Discard the supernatant, very gently as the pellet is very loose.

7. Resuspend the pellet in 4 ml of fresh Neurobasal-N medium with a 5-ml pipet by gently triturating (i.e., pipetting up and down) two or three times. Bring the volume to 12 ml (or the equivalent of 1 embryo/ml) with 2 parts of fresh Neurobasal N medium and 1 part of conditioned medium.

8. Assess cell viability with SYTOX (Support Protocol 2), count cells using a hemacytometer, and plate onto poly-D-lysine coated coverslips inside wells of 12- or 24-well plates at 2×10⁵ cells/well in 700 μl complete Neurobasal N medium for cortical neurons.

9. Incubate plated cells at 37° C. with 4.5% CO₂/95% humidity and monitor CO₂ with a Combustion Test Kit because most electronic panels do not give an accurate reading.

10. Every third day, add 50 μl/well complete Neurobasal-N complete medium. On the sixth day after plating, remove one-quarter of the medium and add the corresponding volume of fresh complete Neurobasal-N medium.

Neurons are ready for coculture (Basic Protocol 5) 10 days after plating.

Transplantation of OL Progenitors into Neonatal Rats—Basic Protocol 6

Neural progenitor cells and their differentiated OL counterparts can be stereotaxically transplanted into the newborn developing rat brain relatively noninvasively as previously described (Snyder et al., 1997; Flax et al., 1998; Espinosa-Jeffrey et al., 2002). Similar results can be obtained with variations on the transplant method that are more suitable depending on the needs of the host brain and the type of study (Yandava et al., 1999; Ourednik et al., 2001, 2002; Park et al., 2002a; Teng et al., 2002; Wakeman et al., 2006; Lee et al., 2007; Redmond et al., 2007). A selection of detailed protocols for neonatal and adult mouse transplantation are described elsewhere (Espinosa de los Monteros et al., 1992, 1993a,b; Yan et al., 2004; Lee et al., 2008; Wakeman et al., 2009; UNIT 2D 3). Upon implantation into the lateral ventricles, donor cells engraft and migrate from the subventricular zone into the host corpus callosum, caudate putamen, and rostral migratory stream (RMS) in much the same manner as host NSC.

Materials

Neonatal rat pup, post-natal day 0 to 5 (P0 to P5)

70% ethanol

Dulbecco's phosphate-buffered saline (DPBS; without calcium or magnesium, e.g., Cellgro, cat. no. 21-031-CV), sterile

Microcentrifuge tube with cell sample (suspended in PBS; may be from various protocols in this example depending on experimental question to be addressed)

Borosilicate glass (Sutter Instrument Co., cat. no. B100-75-15)

Micropipet puller (Sutter Instrument Co., Model P-87)

Aspirator tube assemblies for calibrated microcapillary pipets (Sigma-Aldrich, cat. no. A5177-5EA)

Fiber-optic light source for transillumination

Warming pad

Warm-water glove balloon

Additional reagents and equipment for preparing injection micropipet (Lee et al., 2008)

NOTE: Required materials may vary depending upon the grafting method of choice.

1. Prepare calibrated drawn borosilicate glass micropipet using borosilicate glass and a micropipet puller (Lee et al., 2008).

2. Anesthetize the neonatal rat pup by placing the pup on wet ice for 1.5 to 3 min until the animal no longer retains locomotion or responds to gentle toe and tail pinch.

Carefully monitor the pup and immediately proceed to transplantation.

3. Insert a calibrated, drawn borosilicate glass micropipet into an aspirator tube assembly. Just prior to drawing up the cells, rinse the micropipet by drawing up and then expelling 5 μl of 70% ethanol five times, followed by sterile DPBS ten times to clean the needle.

4. Gently flick sample in microcentrifuge tube prior to filling the needle, wipe the tube with 70% ethanol, and uncap the tube.

5. Slowly draw 4 to 5 μl cell suspension into the micropipet.

6. Loosely secure the head of the anesthetized pup and place directly over the light source to visualize the eyes and bregma.

7. Carefully insert the glass needle into the head at the midline between eye and bregma and slowly inject 2 to 5 μl cell suspension at 5×10⁴ cells/μl into the lateral ventricle of either the left or the right hemisphere. Slowly remove the needle and check for leakage through the needle track. Repeat step 6 into the contralateral hemisphere.

In addition to the lateral ventricles, NSC can also be transplanted into the striatum, the substantia nigra (SN), and corpus callosum (CC; Bjugstad et al., 2005, 2008; Redmond et al., 2007). Upon implantation into the CC of the host, HFB-2050 donor cells recognized by the fluorescent Fast Blue (FB) label migrated along the CC and into the caudate putamen. Precommitted OL can also be placed locally within focal sites of injury to decrease the need for extensive migration.

8. After the injection, warm the pup by placing on a warm-water glove balloon or heating pad to increase the body temperature before returning to the mother.

### Reagents and Solutions

For culture recipes and steps, use sterile tissue culture-grade water. For other purposes, use deionized, distilled water or equivalent in recipes and protocol steps.

Basal Stem Cell Medium (STM-II)

Prepare 1 liter DMEM (low glucose, without glutamine, with sodium pyruvate; Invitrogen, cat. no. 11995-065). Supplement with the following:
5 mg insulin (Sigma, cat. no. 1-5500)
50 mg transferrin (Sigma, cat. no. T-2252)
16.1 mg putrescine (Sigma, cat. no. P-7505)
20 nM (6.29 mg/liter) progesterone (Sigma, cat. no. P-7556)
8 μg sodium selenite (Sigma, cat no. S-5261); add 10 μl/liter of 0.8 mg/ml stock solution in PBS
2.2 g sodium bicarbonate (Fisher, cat. no. S233-500)
1 ml 10,000 U/ml penicillin/10 mg/ml streptomycin (Sigma, cat. no. P-4333) 1 ml 50 mg/ml kanamycin (Sigma, cat. no. K-0254)

Store up to 2 weeks at 4° C.

This medium is used for rat and human NSC. STM-II is a variation of the original STM medium we previously described (Espinosa et al., 2002; UCLA case number 2002-475, formula available by Materials Transfer Agreement). STM-II yields results comparable to those obtained with STM.

Complete Stem Cell Medium II (STMIIc)

Just before use, combine 500 ml basal stem cell medium (STM-II; see recipe) and 500 ml NB-B-27 medium (see recipe).

This medium is used for plating, maintenance, and propagation of NSCs.

OL Specification Medium (OSM-II)

Mix freshly prepared complete stem cell medium (STMIIc; see recipe) with freshly prepared glia defined medium (GDM; see recipe) at a 1:1 (v/v) ratio.

This medium was formerly named OTM (Espinosa-Jeffrey et al., 2002); OSM-II derives from STM-II (see recipe).

Glia Defined Medium (GDM)

Combine 1 liter double-distilled water and one package DMEM/F12 medium (high glucose), then supplement with:
5 mg insulin (Sigma, cat. no. 1-5500)
50 mg transferrin (Sigma, cat. no. T-2252)
16.1 mg putrescine (Sigma, cat. no. P-7505))
2.2 g sodium bicarbonate (Fisher, cat no. S233-500) 4.6 g D-(+)-galactose (Sigma, cat. no. G-0625)
8 μg/ml of sodium selenite (Sigma, cat. no. S-5261): prepare 0.8 mg/ml stock solution in PBS (Sigma, cat. no. P-5368) and add 10 μl of this stock per liter medium
1 ml 50 mg/ml kanamycin (Sigma, cat. no. K-0254)

Filter-sterilize through a 0.22-pm filter.

Prepare fresh.

From Espinosa de los Monteros et al. (1988, 1997).

Neurobasal-B-27 (NB-B-27) Human Neural Stem Cell Proliferation Medium

Prepare 484 ml Neurobasal medium without Normocin, heparin, vitamin A, or LIF (Invitrogen, cat. no. 21103-049). Store up to 2 weeks at 4° C. Just before use in preparing STMIIc medium (see recipe), supplement with:
10 ml B-27 supplement without vitamin A (Invitrogen, cat. no. 12587-010) 5 ml GlutaMAX (Invitrogen, cat. no. 35050-061)
8 μg/ml heparin (Sigma, cat. no. H-3149)
2 ng/ml basic fibroblast growth factor (bFGF; Invitrogen, cat. no. 13256-029) 10 ng/ml leukemia inhibitory factor (LIF; Millipore, cat. no. LIF-1010)

Neurobasal-N Medium for Cortical Neurons, Complete
1 liter Neurobasal medium (Invitrogen, cat. no. 21103-049) supplemented with: 5 mg insulin (Sigma, cat. no. 1-5500))
50 mg transferrin (Sigma, cat. no. T-2252)
8 μg sodium selenite (Sigma, cat. no. S-5261): prepare 0.8 mg/ml stock solution in PBS (Sigma, cat. no. P-5368) and add 10 pl of this stock per liter medium
2.2 g sodium bicarbonate (Fisher, cat. no. S233-500)
1 ml kanamycin (Sigma, cat. no. K-0254)

Just before using, add the following to the supplemented Neurobasal-N to make the complete medium:
1:50 (v/v) B-27 supplement with vitamin A (Invitrogen, cat. no. 17504-044) 20 ng/ml recombinant basic bFGF (Invitrogen, cat. no. 13256-029)

OLDEM Medium

Prepare glia defined medium (GDM; see recipe), but omit transferrin.

Poly-D-Lysine Coated Wells/Plates/Coverslips

Prepare a stock solution by dissolving 100 mg poly-D-lysine in 100 ml water and filter sterilize through a 0.22-pm filter. Store in 5-ml aliquots at −20° C. When ready to use, dilute 1 part stock solution with 9 parts water to prepare 100 μg/ml working solution. Fill tissue culture dishes or wells with the working solution (and/or place coverslips to be coated into wells of 12- or 24-well plate) and incubate 1 hr in a humidified 37° C., 5% CO₂ incubator, then remove solution by vacuum aspiration and allow surface to dry. Store coated tissue culture ware up to 3 months at 4° C. Use diluted solutions only once, but unused diluted aliquots can be stored up to 3 months at 4° C.

Tris-Buffered Saline (TBS)
2.42 g/liter Tris base
29.22 g/liter NaCl
Adjust pH to 7.5 with HCl

Store at room temperature up to 1 year, under sterile conditions.

### Commentary

Background Information

The described culturing system allows for the production of relatively homogeneous primary OL cultures in adequate numbers for cryopreservation. These cell stocks can be used for basic research in further in vitro studies. Moreover, these cells are never exposed to animal or human sera, and therefore remain as suitable candidates for cell replacement therapies in developmental disorders of the central nervous system (CNS) as well as neurodegenerative diseases.

Parameters

We want to emphasize that fate restriction towards commitment from NSC to OLP (as defined in Basic Protocol 3) becomes irreversible after NSCs have been in OSM for at least 20 hr in either two- or three-dimensional cultures. Therefore, the progeny of these cells will define a homogeneous OLP population, ideal for biochemical, toxicological, and pharmacological studies, and also serve as an appropriate and reproducible source of committed cells to be used in cell therapy studies.

Monitor the concentration of CO₂ with a Combustion Test Kit, as most electronic panels do not provide an accurate reading. The proper lineage progression relies on precise control of CO₂ to maintain a pH that should remain accurate and controlled.

Troubleshooting

Human NSC are more fragile than their rodent counterparts; therefore, we recommend dissociation protocols that favor as little mechanical stress as possible. In our hands, enzymatic dissociation with 2 to 4 ml Accutase (Millipore) at 37° C. for 3 to 5 min or light mechanical trituration through an 18-G needle (three to five times) is sufficient to dissociate hNSC into single cells and small (2- to 6-cell) clusters.

Results

OLPs obtained utilizing this system are plated on anti-PSA/NCAM plates and will attain a bipolar morphology if maintained in freshly supplemented OSM. Cells can also be plated directly onto plastic (tissue-culture grade). The morphology in that case may look more flattened or fibroblastic, but if maintained in fresh OSM, the early markers such as Olig2, Tf, PDGF-R, and NG2 will be expressed. At this stage, cells are still highly motile but will migrate less if plated onto poly-D-lysine. During this time, cells attain a more mature phenotype that truly represents their in vivo counterparts.

Our culture media formulation includes the minimum and sufficient nutrients to support a given developmental stage; thus, cells cannot be kept indefinitely under these conditions because the substratum dictates the organization of the molecules on the cell membrane and poly-D-lysine confers a more permanent adhesion to the cells. Consequently, they would have the tendency to mature based on the signals coming from the cell membrane-substrate interaction (Linnemann and Bock, 1989; Mauro et al., 1994). Unfortunately, cells will not survive or remain healthy if maintained in unreplenished OSM as 2-D cultures, due to a lack of nutrients to support their transition to the next developmental stage. These cells will survive well if fed with OSM to renew the growth factors. The same concept applies to the transition to more mature OL stages. The nutrients and substrate together contribute to support cell signaling that will result in the formation of multiple cell processes followed by the synthesis of myelin components and their organization for membrane formation.

Time Considerations

The initial dissection and preparation of the primary cell suspension takes 2 hr. From the moment cells are plated on anti-PSA-NCAM (if fed regularly with fresh humoral factors), 100-mm dishes can be confluent within 3 to 4 days. Thus, generating 20 vials of rat NSCs for cryostorage would take 16 days. The generation of OLP from rNSC takes 24 hr; however, generating OLP in high numbers (15 vials) for storage would take 4 to 6 weeks. Lineage progression of rat OL towards more mature phenotypes takes 48 hr in the specific culture medium (GDM or OLDEM). In addition, OL will still proliferate in GDM, but at a much slower rate. Both GDM and OLDEM media are favorable to protein synthesis but less favorable for cell proliferation.

Previously isolated ES cells and their NSC derivatives will need a longer period of time to provide high numbers of NSC for frozen stocks. This time will vary depending on the origin of the sample. We have had similar success directing NSC from several species, utilizing the same chemically defined media; however, incubation times may need to be increased for full maturation in higher-order mammals, such as primates. Induced cells lose NSC characteristics and acquire OLP features within 72 hr, yet their cell cycle is much slower, and, therefore, it would be necessary to propagate these cells 8 to 10 weeks to be able to create a healthy stock (six to eight vials) of human OLP. Previously established NSC lines (Snyder et al., 1992) can also be propagated and specified into the OL phenotype using the system described here.

### LITERATURE CITED

Ahn, S. M., Byun, K., Kim, D., Lee, K., Yoo, J. S., Kim, S. U., Jho, E. H., Simpson, R. J., and Lee, B. 2008. Olig2-induced neural stem cell differentiation involves downregulation of Wnt signaling and induction of Dickkopf-1 expression. PLoS ONE 3:e3917.
Arenander, A. T. and De Vellis, J. 1995. Early-response gene expression in glial cells. in Neuroglia (H. Kettenmann and B. R. Ransom, eds.) pp. 510-522. Oxford University Press, New York.
Avellana-Adalid, V., Nait-Oumesmar, B., Lachapelle, F., and Baron-Van Evercooren, A. 1996. Expansion of rat oligodendrocyte progenitors into proliferative “oligospheres” that retain differentiation potential. J. Neurosci. Res. 45:558-570.
Bjugstad, K. B., Redmond, D. E. Jr., Teng, Y. D., Elsworth, J. D., Roth, R. H., Blanchard, B. C., Snyder, E. Y., and Sladek, J. R. Jr. 2005. Neural stem cells implanted into MPTP-treated monkeys increase the size of endogenous tyrosine hydroxylase-positive cells found in the striatum: A return to control measures. Cell Transplant. 14:183-192.
Bjugstad, K. B., Teng, Y. D., Redmond, D. E. Jr., Elsworth, J. D., Roth, R. H., Cornelius, S. K., Snyder, E. Y., and Sladek, J. R. Jr. 2008. Human neural stem cells migrate along the nigrostriatal pathway in a primate model of Parkinson's disease. Exp. Neurol. 211:362-369.
Bottenstein, J. E. and Sato, G. H. 1979. Growth of a rat neuroblastoma cell line in serum-free supplemented medium. Proc. Natl. Acad. Sci. U.S.A. 76:514-517.
Chattopadhyay, N., Espinosa-Jeffrey, A., TfeltHansen, J., Yano, S., Bandyopadhyay, S., Brown, E. M., and de Vellis, J. 2008. Calcium receptor expression and function in oligodendrocyte commitment and lineage progression: Potential impact on reduced myelin basic protein in CaR-null mice. J. Neurosci. Res. 86:2159-2167.
De Filippis, L., Lamorte, G., Snyder, E. Y., Malgaroli, A., and Vescovi, A. L. 2007. A novel, immortal, and multipotent human neural stem cell line generating functional neurons and oligodendrocytes. Stem Cells 25:2312-2321.
Espinosa de los Monteros, A., Roussel, G, Neskovic, N. M. and Nussbaum, J. L. 1988. A chemically defined medium for the culture of mature oligodendrocytes. J. Neurosci. Res. 19:202-211.
Espinosa de los Monteros, A., Zhang, M., Gordon, M., Aymie, M., and de Vellis, J. 1992. Transplantation of cultured premyelinating oligodendrocytes into normal and myelin-deficient rat brain. Dev. Neurosci. 14:98-104.
Espinosa de los Monteros, A., Zhang, M.-S., and de Vellis, J. 1993a. O2A progenitor cells transplanted into the neonatal rat brain develop Into oligodendrocytes but not astrocytes. Proc. Natl. Acad. Sci. U.S.A. 90:50-54.
Espinosa de los Monteros, A., Bernard, R., Tiller, B., Rouget, P., and de Vellis, J. 1993b. Grafting of fast blue labeled glial cells into neonatal rat brain: Differential survival and migration among cell types. Int. J. Dev. Neurosci. 11:625-639.
Espinosa de los Monteros, A. and de Vellis, J. 1996. Vulnerability of oligodendrocytes to environmental insults: Potential for recovery. In The Role of Glia in Neurotoxicity (M. Aschner and H. K. Kimelberg, eds.) pp. 15-46. CRC Press, Boca Raton, Fla.
Espinosa de los Monteros, A., Yuan, J., McCartney, D., Madrid, B. R., Cole, R., Kanfer, J. N., and de Vellis, J. 1997. Acceleration of the maturation of oligodendroblasts into oligodendrocytes and enhancement of their myelinogenic properties by a chemically defined medium. Dev. Neurosci. 19:297-311.
Espinosa-Jeffrey, A., Becker-Catania, S., Zhao, P. M., Cole, R., Edmond, J. and de Vellis, J. 2002. Phenotype specification and development of oligodendrocytes and neurons from rat stem cell cultures using two chemically defined media. J. Neurosci. Res. 69 (8):810-825. Selective specification of CNS stem cells into oligodendroglial or neuronal cell lineage: cell culture and transplant studies
Flax, J. D., Aurora, S., Yang, C., Simonin, C., Wills, A. M., Billinghurst, L. L., Jendoubi, M., Sidman, R. L., Wolfe, J. H., Kim, S. U., and Snyder, E. Y. 1998. Engraftable human neural stem cells respond to developmental cues, replace neurons, and express foreign genes. Nat. Biotechnol. 16:1033-1039.
Kim, H. T., Kim, I. S., Lee, I. S., Lee, J. P., Snyder, E. Y., and Park, K. I. 2006. Human neurospheres derived from the fetal central nervous system are regionally and temporally specified but are not committed. Exp. Neurol. 199:222-235.
Kim, S. U. 2004. Human neural stem cells genetically modified for brain repair in neurological disorders. Neuropathology 24:159-171.
Kim, S. U., Nagai, A., Nakagawa, E., Choi, H. B., Bang, J. H., Lee, H. J., Lee, M. A., Lee, Y. B., and Park, I.H.2008. Production and characterization multipotent differentiation property. In Methods in Molecular Biology, Vol. 438: Neural Stem Cells, 2nd ed. (L. P. Weiner, ed.) pp. 103-121. Humana Press, Totowa, N. J.
Larsen, E. C., Kondo, Y., Fahrenholtz, C. D., and Duncan, I. D. 2008. Generation of cultured oligodendrocyte progenitor cells from rat neonatal brains. Curr. Protoc. Stem Cell Biol. 6:2 D.1.1-2D.1.13.
Lee, H. J., Kim, K. S., Kim, E. J., Choi, H. B., Lee, K. H., Park, I. H., Ko, Y., Jeong, S. W., and Kim, S. U. 2007. Brain transplantation of immortalized human neural stem cells promotes functional recovery in mouse intracerebral hemorrhage stroke model. Stem Cells 25:1204-1212.
Lee, J. P., McKercher, S., Müller, F. J., and Snyder, E. Y. 2008. Neural stem cell transplantation in mouse brain. Curr. Protoc. Neurosci. 42:3.10.1-3.10.23.
Linnemann, D. and Bock, E. 1989. Cell adhesion molecules in neural development. Dev. Neurosci. 11:149-173.
Louis, J. C., Magal, E., Muir, D., Manthorpe, M., and Varon, S. 1992. CG4, a new bipotential glial cell line from rat brain, is capable of differentiating in vitro either mature oligodendrocytes or type-2 astrocytes. J. Neurosci. Res. 31:193-204.
Mauro, V. P., Wood, I. C., Krushel, C., Crossin, K. L., and Edelman, G. M. 1994. Cell adhesion alters gene transcription in chicken embryo brain cells and mouse embryonal carcinoma cells. Proc. Natl. Acad. Sci. U.S.A. 91:2868-2872.
Muller, F. J., Snyder, E. Y., and Loring, J. F. 2006. Gene therapy: Can neural stem cells deliver? Nat. Rev. Neurosci. 7:75-84.
Neman, J. and De Vellis, J., eds. 2008. Handbook of Neurochemistry and Molecular Neurobiology: Myelinating Cells in the Central Nervous System—Development, Aging, and Disease. Springer, New York.
Ourednik, V., Ourednik, J., Flax, J. D., Zawada, W. M.,
Hutt, C., Yang, C., Park, K. I., Kim, S. U., Sidman, R. L., Freed, C. R., and
Snyder, E. Y. 2001. Segregation of human neural stem cells in the developing primate forebrain. Science 293:1820-1824.
Ourednik, J., Ourednik, V., Lynch, W. P., Schachner, M., and Snyder, E. Y. 2002. Neural stem cells display an inherent mechanism for rescuing dysfunctional neurons. Nat. Biotechnol. 20:1103-1110.
Palmer, T. D., Schwartz, P. H., Taupin, P., Kaspar, B., Stein, S. A., and Gage, F. H. 2001. Cell culture: Progenitor cells from human brain after death. Nature 411:42-43.
Park, K. I., Teng, Y. D., and Snyder, E. Y. 2002a. The injured brain interacts reciprocally with neural stem cells supported by scaffolds to reconstitute lost tissue. Nat. Biotechnol. 20:1111-1117.
Park, K. I., Ourednik, J., Ourednik, V., Taylor, R. M., Aboody, K. S., Auguste, K. I., Lachyankar, M. B., Redmond, D. E., and Snyder, E. Y. 2002b. Global gene and cell replacement strategies via stem cells. Gene Ther. 9:613-624.
Redmond, D. E. Jr., Bjugstad, K. B., Teng, Y. D., Ourednik, V., Ourednik, J., Wakeman, D. R., Parsons, X. H., Gonzalez, R., Blanchard, B. C., Kim, S. U., Gu, Z., Lipton, S. A., Markakis, E. A., Roth, R. H., Elsworth, J. D., Sladek, J. R. Jr., Sidman, R. L., and Snyder, E. Y. 2007. Behavioral improvement in a primate Parkinson's model is associated with multiple homeostatic effects of human neural stem cells. Proc. Natl. Acad. Sci. U.S.A. 104:12175-12180.
Saneto, R. P. and de Vellis, J. 1985. Characterization of cultured rat oligodendrocytes proliferating in a serum-free chemically defined medium. Proc. Natl. Acad. Sci. U.S.A. 82:3509-3513.
Schwartz, P. H., Bryant, P. J., Fuja, T. J., Su, H., O'Dowd, D. K., and Klassen, H.2003. Isolation and characterization of neural progenitor cells from post-mortem human cortex. J. Neurosci. Res. 74:838-851.
Snyder, E. Y., Deitcher, D. L., Walsh, C., ArnoldAldea, S., Hartwieg, E. A., and Cepko, C. L. 1992. Multipotent neural cell lines can engraft and participate in development of mouse cerebellum. Cell 68:33-51.
Snyder, E. Y., Yoon, C., Flax, J. D., and Macklis, J. D. 1997. Multipotent neural precursors can differentiate toward replacement of neurons undergoing targeted apoptotic degeneration in adult mouse neocortex. Proc. Natl. Acad. Sci. U.S.A. 94:11663-11668.
Svendsen, C. N., Caldwell, M. A., and Ostenfeld, T. 1999. Human neural stem cells: Isolation, expansion and transplantation. Brain Pathol. 9:499-513.
Teng, Y. D., Lavik, E. B., Qu, X., Park, K. I., Ourednik, J., Zurakowski, D., Langer, R., and Snyder, E. Y. 2002. Functional recovery following traumatic spinal cord injury mediated by a unique polymer scaffold seeded with neural stem cells. Proc. Natl. Acad. Sci. U.S.A. 99:3024-3029.
Villa, A., Snyder, E. Y., Vescovi, A., and Mart'inezSerrano, A. 2000. Establishment and properties of a growth factor-dependent, perpetual neural stem cell line from the human CNS. Exp. Neurol. 161:67-84.
Wakeman, D. R., Crain, A. C., and Snyder, E. Y. 2006. Large animal models are critical for rationally advancing regenerative therapies. Regenerative Med. 1:405-413.
Wakeman, D. R., Hofmann, M. R., Teng, Y. D., and Snyder, E. Y. 2009. Derivation, expansion, and characterization of human fetal forebrain neural stem cells. In Human Cell Culture: Adult Stem Cells: Vol. 7. (J. R. Masters and B. O. Palsson, eds.). Springer, Dordrecht, The Netherlands.
Williams, W. C. 2nd and Gard, A. L. 1997. In vitro death of jimpy oligodendrocytes: Correlation with onset of DM-20/PLP expression and resistance to oligodendrogliotrophic factors. J. Neurosci. Res. 50:177-189.
Wysocki, L. J. and Sato, V. L. 1978. Panning for lymphocytes: A method for cell selection. Proc. Natl. Acad. Sci. U.S.A. 75:2844-2848.
Yan, J., Welsh, A. M., Bora, S. H., Snyder, E. Y., and Koliatsos, V. E. 2004. Differentiation and tropic/trophic effects of exogenous neural precursors in the adult spinal cord. J. Comp. Neurol. 480:101-114.
Yandava, B. D., Billinghurst, L. L., and Snyder, E. Y. 1999. Global cell replacement is feasible via neural stem cell transplantation: Evidence from the dysmyelinated shiverer mouse brain. Proc. Natl. Acad. Sci. U.S.A. 96:7029-7034.
Yang, Z., Watanabe, M., and Nishiyama, A. 2005. Optimization of oligodendrocyte progenitor cell culture method for enhanced survival. J. Neurosci. Methods 149:50-56.
Zhang, S. C., Lundberg, C., Lipitz, D., O'Connor, L. T., and Duncan, I. D. 1998. Generation of oligodendroglial progenitors from neural stem cells. J. Neurocytol. 27:475-489.

### Example 2

Embryoid bodies from cell line hiPS21 were obtained from Dr. Bill Lowry from the Department of Molecular and Developmental Biology at UCLA. The culture medium was removed without disturbing the EBs (removing most of the volume). Fresh iPESSOLM (Table 5 above) was slowly delivered to each well. On the second and third days, the same procedure was performed. On day 4, EBs were transferred to 12-well plates coated with either laminin or anti-IgM antibody or anti-PSA N-CAM, A2B5 or O4 antibodies as described (Espinosa et al. 2002, 2009). EB's adhered to the substratum selectively when antibodies were used or non-selectively on either glass or laminin.

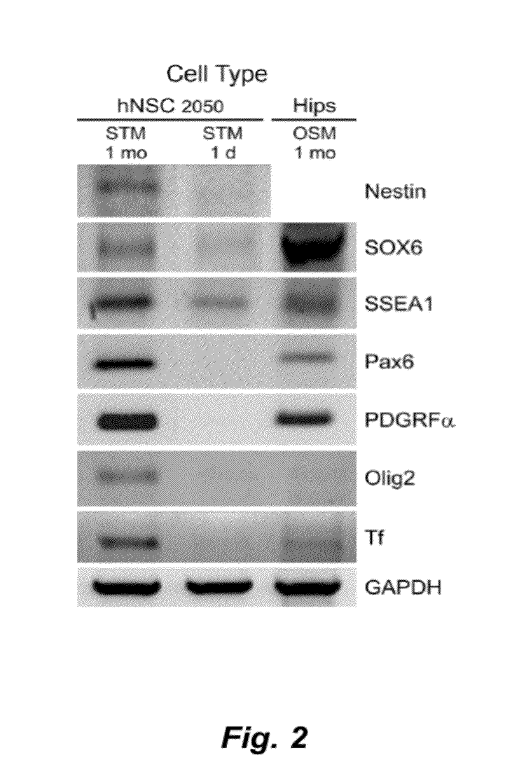

FIG. 2 shows the expression of neural stem cell markers by the neural stem cell line 2050 cultured in STM for 1 month or 1 day (lanes 1 and 2) and hIPS-21 cultured 1 month in iP/ESSOLM lane 3.

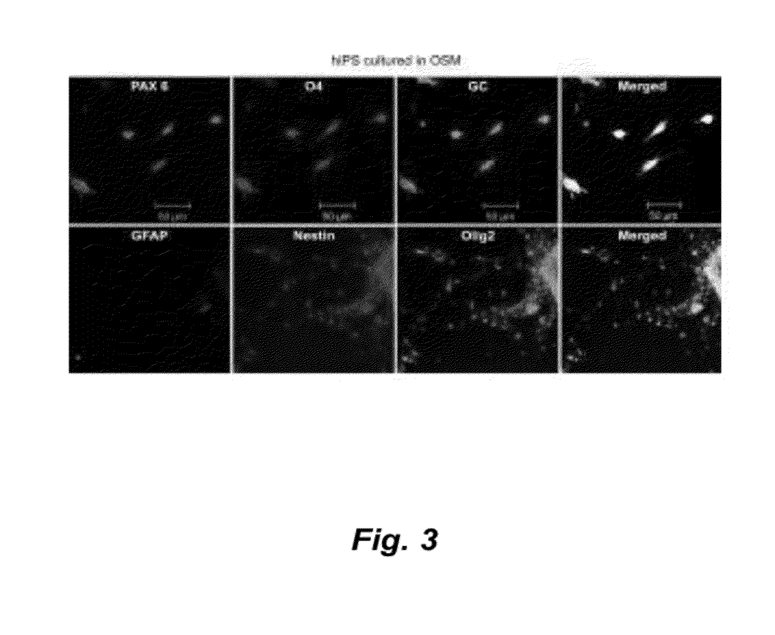

In order to determine that these cells expressed oligodendrocyte-specific markers, we examined the cultures with triple immunofluorescence for neural (Pax 6 and nestin) and oligodendrocyte markers such as Olig2, sulfatides detected by the antibody O4, and galactocerebrosides (GC). See FIG. 3. These cultures did not contain astrocytes as shown by the absence of glial fibrilary acidic protein (GFAP).

1. A nutrient formula for use in propagating stem cells, the nutrient formula comprising:
Hydroxybutyrate
Hypoxanthine-Na
i-Inositol
L-Alanine
L-Arginine hydrochloride
L-Asparagine-H2O
L-Cysteine-HCl—H2O
L-Cystine-2HCl
L-Glutamine
L-Histidine-HCl—H2O
L-Isoleucine
L-Leucine
L-Lysine hydrochloride
L-Methionine
L-Phenylalanine
L-Proline
L-Serine
L-Threonine
L-Tryptophan
L-Tyrosine-2Na-2H2O
L-Valine
Lipoic acid
Creatine
bFGF
EGF
Insulin
Transferrin
Pen/Strep

2. The nutrient formula of claim 1, wherein the nutrient formula comprises:

| | Molarity | |
|---|---|---|
| Components | (mM) | mg/L |
| Hydroxybutyrate | 0.5 | 80.1 |
| Hypoxanthine-Na | 0.0075 | 1.192 |
| i-Inositol | 0.055 | 9.9 |
| L-Alanine | 0.025 | 2.225 |
| L-Arginine hydrochloride | 0.549 | 115.839 |
| L-Asparagine-H2O | 0.025 | 3.75 |
| L-Cysteine-HCl—H2O | 0.05 | 8.8 |
| L-Cystine-2HCl | 0.15 | 46.95 |
| L-Glutamine | 2 | 292 |
| L-Histidine-HCl—H2O | 0.175 | 36.75 |
| L-Isoleucine | 0.609 | 79.779 |
| L-Leucine | 0.6265 | 82.0715 |
| L-Lysine hydrochloride | 0.6485 | 118.6755 |
| L-Methionine | 0.1585 | 23.6165 |
| L-Phenylalanine | 0.3075 | 50.7375 |
| L-Proline | 0.075 | 8.625 |
| L-Serine | 0.325 | 34.125 |
| L-Threonine | 0.2635 | 31.3565 |
| L-Tryptophan | 0.0611 | 12.4644 |
| L-Tyrosine-2Na—2H2O | 0.306 | 68.85 |
| L-Valine | 0.6275 | 73.4175 |
| Lipoic acid | 0.000255 | 0.05253 |
| Creatine | 0.0128 | 1000 mg/L |
| bFGF | | 20 ng/ml |
| EGF | | 10 ng/ml |
| Insulin | | 10 ug/ml |
| Transferrin | | 50 mg/L |
| Pen/Strep | | 0.5 ml/L |

3. The nutrient formula of claim 4, wherein the nutrient formula additionally comprises 1 ml/L B-27 with Retinoic acid.

4. A culture medium for use in propagating stem cells, the culture medium comprising:
Biotin
Calcium chloride
Choline chloride
Cupric sulfate
D-Calcium pantothenate
D-Glucose
Ethanolamine
Ferric nitrate
Ferrous sulfate
Folic acid
Fumaric acid
Glycine
Hepes
Hydroxybutyrate
Hypoxanthine-Na
i-Inositol
L-Alanine
L-Arginine hydrochloride
L-Aspartic acid
L-Asparagine-H2O
L-Cysteine-HCl—H2O
L-Cystine-2HCl
L-Glutamic acid
L-Glutamine
L-Histidine-HCl-H2O
L-Isoleucine
L-Leucine
L-Lysine hydrochloride
L-Methionine
L-Phenylalanine
L-Proline
L-Serine
L-Threonine
L-Tryptophan
L-Tyrosine-2Na-2H20
L-Valine
Linoleic acid
Lipoic acid
Magnesium chloride
Magnesium sulfate
Niacinamide
Phenol red
Potassium chloride
Putrescine-2HCl
Pyridoxine hydrochloride
Pyruvic acid
Riboflavin
Sodium chloride
Sodium bicarbonate
Sodium phosphate, mono.
Sodium phosphate, dibas.
Sodium pyruvate
Thiamine hydrochloride
Thymidine
Vitamin B12
Zinc sulfate
Creatine
B-27
bFGF
EGF
Kanamycin
Insulin
Transferrin
Pen/Strep

5. The culture medium of claim 4, wherein the culture medium comprises:

| | Molarity | |
|---|---|---|
| Components | (mM) | mg/L |
| Biotin | 0.000011075 | 0.002702 |
| Calcium chloride | 1.14975 | 127.6223 |
| Choline chloride | 0.0552 | 7.728 |
| Cupric sulfate | 0.0000038 | 0.00095 |
| D-Calcium | 0.00555 | 2.64735 |
| pantothenate | | |
| D-Glucose | 17 | 3060 |
| Ethanolamine | 0.125 | 7.625 |
| Ferric nitrate | 0.000154 | 0.062216 |
| Ferrous sulfate | 0.001125 | 0.31275 |
| Folic acid | 0.00689 | 3.03849 |
| Fumaric acid | 0.25 | |
| Glycine | 0.26835 | 20.12625 |
| Hepes | 3.75 | |
| Hydroxybutyrate | 0.25 | |
| Hypoxanthine-Na | 0.01125 | |
| i-Inositol | 0.0625 | 11.25 |
| L-Alanine | 0.0375 | 3.3375 |
| L-Arginine | 0.624 | 131.664 |
| hydrochloride | | |
| L-Asparagine-H2O | 0.0375 | 5.625 |
| L-Aspartic acid | 0.025 | 3.325 |
| L-Cysteine-HCl—H2O | 0.075 | 13.2 |
| L-Cystine-2HCl | 0.125 | 39.125 |
| L-Glutamic Acid | 0.025 | 3.675 |
| L-Glutamine | 2.25 | 328.5 |
| L-Histidine-HCl—H2O | 0.16245 | 34.1145 |
| L-Isoleucine | 0.5125 | 67.1375 |
| L-Leucine | 0.52075 | 68.21825 |
| L-Lysine | 0.5735 | 104.9505 |
| hydrochloride | | |
| L-Methionine | 0.137 | 20.413 |
| L-Phenylalanine | 0.26125 | 43.10625 |
| L-Proline | 0.1125 | 12.9375 |
| L-Serine | 0.2875 | 30.1875 |
| L-Threonine | 0.17625 | 20.97375 |
| L-Tryptophan | 0.05255 | 10.7202 |
| L-Tyrosine-2Na—2H2O | 0.26 | 58.5 |
| L-Valine | 0.5395 | 63.1215 |
| Linoleic acid | 0.000075 | 0.021 |
| Lipoic acid | 0.00037025 | 0.076272 |
| Magnesium chloride | 0.225 | 21.375 |
| Magnesium sulfate | 0.50825 | 60.99 |
| Niacinamide | 0.0206 | 2.5132 |
| Phenol red | 0.01805 | 7.1839 |
| Potassium chloride | 4.435 | 332.625 |
| Putrescine-2HCl | 0.00037575 | 0.060496 |
| Pyridoxine | 0.0123 | 2.5338 |
| hydrochloride | | |
| Pyruvic acid | 0.25 | |
| Riboflavin | 0.00070525 | 0.265174 |
| Sodium chloride | 118.085 | 6848.93 |
| Sodium bicarbonate | 36.375 | 3055.5 |
| Sodium phosphate, | 0.33975 | |
| mono. | | |
| Sodium phosphate, | 0.125 | |
| dibas. | | |
| Sodium pyruvate | 1.125 | 123.75 |
| Thiamine | 0.00775 | 2.61175 |
| hydrochloride | | |
| Thymidine | 0.001125 | 0.27225 |
| Vitamin B12 | 0.000625 | 0.846875 |
| Zinc sulfate | 0.001125 | 0.324 |
| Creatine | 3.355 | 2000 |
| B-27 | 0.038194444 | |
| bFGF | | |
| EGF | | |
| Kanamycin | | |
| Insulin | | 5 |
| Transferrin | | 50 |
| Pen/Strep | | |

6. The culture medium of claim 4, wherein the culture medium comprises:

| | Molarity | |
|---|---|---|
| Components | (mM) | mg/L |
| Biotin | 0.000011075 | 0.002702 |
| Calcium chloride | | 138.75 |
| Choline chloride | 0.0552 | 7.728 |
| Cupric sulfate | 0.0000038 | 0.00095 |
| D-Calcium | 0.00555 | 2.64735 |
| pantothenate | | |
| D-Glucose | | 2970 |
| Ethanolamine | 0.125 | 7.625 |
| Ferric nitrate | 0.000154 | 0.062216 |
| Ferrous sulfate | 0.001125 | 0.31275 |
| Folic acid | 0.00689 | 3.03849 |
| Fumaric acid | 0.25 | 0 |
| Glycine | | 21.54 |
| Hepes | | 1785 |
| Hydroxybutyrate | | 80.1 |
| Hypoxanthine-Na | | 1.192 |
| i-Inositol | | 9.9 |
| L-Alanine | 0.0375 | 3.3375 |
| L-Arginine | 0.624 | 131.664 |
| hydrochloride | | |
| L-Asparagine-H2O | 0.0375 | 5.625 |
| L-Aspartic acid | 0.025 | 3.325 |
| L-Cysteine-HCl—H2O | 0.075 | 13.2 |
| L-Cystine-2HCl | 0.125 | 39.125 |
| L-Glutamic Acid | 0.025 | 3.675 |
| L-Glutamine | 2.25 | 328.5 |
| L-Histidine-HCl—H2O | 0.16245 | 34.1145 |
| L-Isoleucine | 0.5125 | 67.1375 |
| L-Leucine | 0.52075 | 68.21825 |
| L-Lysine | 0.5735 | 104.9505 |
| hydrochloride | | |
| L-Methionine | 0.137 | 20.413 |
| L-Phenylalanine | 0.26125 | 43.10625 |
| L-Proline | 0.1125 | 12.9375 |
| L-Serine | 0.2875 | 30.1875 |
| L-Threonine | 0.17625 | 20.97375 |
| L-Tryptophan | 0.05255 | 10.7202 |
| L-Tyrosine-2Na—2H2O | 0.26 | 58.5 |
| L-Valine | 0.5395 | 63.1215 |
| Linoleic acid | 0.000075 | 0.021 |
| Lipoic acid | 0.00037025 | 0.076272 |
| Magnesium chloride | 0.225 | 21.375 |
| Magnesium sulfate | 0.50825 | 60.99 |
| Niacinamide | 0.0206 | 2.5132 |
| Phenol red | | 6.8854 |
| Potassium chloride | 4.435 | 332.625 |
| Putrescine-2HCl | 0.00037575 | 0.060496 |
| Pyridoxine | 0.0123 | 2.5338 |
| hydrochloride | | |
| Pyruvic acid | 0.25 | 0 |
| Riboflavin | 0.00070525 | 0.265174 |
| Sodium chloride | 118.085 | 6848.93 |
| Sodium bicarbonate | | 3670.8 |
| Sodium phosphate, | | 96.489 |
| mono. | | |
| Sodium phosphate, | | 34.50 |
| dibas. | | |
| Sodium pyruvate | | 137.5 |
| Thiamine | | 3.00667 |
| hydrochloride | | |
| Thymidine | 0.001125 | 0.27225 |
| Vitamin B12 | 0.000625 | 0.846875 |
| Zinc sulfate | 0.001125 | 0.324 |
| Creatine | | 1000 |
| B-27+ Retinoic acid | | 1 ml/L |
| bFGF | | 20 ng/ml |
| EGF | | 10 ng/ml |
| Kanamycin | | 1 ml/L |
| Insulin | | 10 |
| Transferrin | | 100 |
| Pen/Strep | | 1 ml/L |
| Normocin | | 1 ml/L |

7. The nutrient formula of claim 1, wherein the nutrient formula comprises one or more components selected from the group consisting of additional insulin, insulin-like growth factor-1 (IGF-1), insulin-like growth factor-2 (IGF-2).

8. A cell culture for propagating stem cells, wherein the cell culture comprises:
stem cells; and
the nutrient formula of claim 1.

9. A nutrient formula for use in inducing the specification of neural stem cells (NSC) to the oligodendrocyte (OL) phenotype, the nutrient formula comprising:
Hydroxybutyrate
Hypoxanthine-Na
i-Inositol
L-Alanine
L-Arginine
hydrochloride
L-Asparagine-H2O
L-Aspartic acid
L-Cysteine-HCl—H2O
L-Cystine-2HCl
L-Glutamic Acid
L-Glutamine
L-Histidine-HCl—H2O
L-Isoleucine
L-Leucine
L-Lysine
hydrochloride
L-Methionine
L-Phenylalanine
L-Proline
L-Serine
L-Threonine
L-Tryptophan
L-Tyrosine-2Na-2H2O
L-Valine
Linoleic acid
Lipoic acid
Creatine
B-27
Transferrin

10. The nutrient formula of claim 9, wherein the nutrient formula comprises:

| | Molarity | Formula | |
|---|---|---|---|
| Components | (mM) | Weight | mg/L |
| Hydroxybutyrate | 0.25 | | |
| Hypoxanthine-Na | 0.01125 | | |
| i-Inositol | 0.0625 | 180 | 11.25 |
| L-Alanine | 0.0375 | 89 | 3.3375 |
| L-Arginine | 0.624 | 211 | 131.664 |
| hydrochloride | | | |
| L-Asparagine-H2O | 0.0375 | 150 | 5.625 |
| L-Aspartic acid | 0.025 | 133 | 3.325 |
| L-Cysteine-HCl—H2O | 0.075 | 176 | 13.2 |
| L-Cystine-2HCl | 0.125 | 313 | 39.125 |
| L-Glutamic Acid | 0.025 | 147 | 3.675 |
| L-Glutamine | 2.25 | 146 | 328.5 |
| L-Histidine-HCl—H2O | 0.16245 | 210 | 34.1145 |
| L-Isoleucine | 0.5125 | 131 | 67.1375 |
| L-Leucine | 0.52075 | 131 | 68.21825 |
| L-Lysine | 0.5735 | 183 | 104.9505 |
| hydrochloride | | | |
| L-Methionine | 0.137 | 149 | 20.413 |
| L-Phenylalanine | 0.26125 | 165 | 43.10625 |
| L-Proline | 0.1125 | 115 | 12.9375 |
| L-Serine | 0.2875 | 105 | 30.1875 |
| L-Threonine | 0.17625 | 119 | 20.97375 |
| L-Tryptophan | 0.05255 | 204 | 10.7202 |
| L-Tyrosine-2Na—2H2O | 0.26 | 225 | 58.5 |
| L-Valine | 0.5395 | 117 | 63.1215 |
| Linoleic acid | 0.000075 | 280 | 0.021 |
| Lipoic acid | 0.00037025 | 206 | 0.076272 |
| Creatine | 0.0064 | | 0.860 |
| B-27 | 0.038194444 | | |
| Transferrin | | | 25 mg/L |

11. The nutrient formula of claim 9, wherein the nutrient formula comprises:

| | Molarity | Formula | |
|---|---|---|---|
| Components | (mM) | Weight | mg/L |
| Hydroxybutyrate | | | 35.5 |
| Hypoxanthine-Na | | | 1.788 |
| i-Inositol | 0.0625 | 180 | 11.25 |
| L-Alanine | 0.0375 | 89 | 3.3375 |
| L-Arginine | 0.624 | 211 | 131.664 |
| hydrochloride | | | |
| L-Asparagine-H2O | 0.0375 | 150 | 5.625 |
| L-Aspartic acid | 0.025 | 133 | 3.325 |
| L-Cysteine-HCl—H2O | 0.075 | 176 | 13.2 |
| L-Cystine-2HCl | 0.125 | 313 | 39.125 |
| L-Glutamic Acid | 0.025 | 147 | 3.675 |
| L-Glutamine | 2.25 | 146 | 328.5 |
| L-Histidine-HCl—H2O | 0.16245 | 210 | 34.1145 |
| L-Isoleucine | 0.5125 | 131 | 67.1375 |
| L-Leucine | 0.52075 | 131 | 68.21825 |
| L-Lysine | 0.5735 | 183 | 104.9505 |
| hydrochloride | | | |
| L-Methionine | 0.137 | 149 | 20.413 |
| L-Phenylalanine | 0.26125 | 165 | 43.10625 |
| L-Proline | 0.1125 | 115 | 12.9375 |
| L-Serine | 0.2875 | 105 | 30.1875 |
| L-Threonine | | 119 | 42.3937 |
| L-Tryptophan | 0.05255 | 204 | 10.7202 |
| L-Tyrosine-2Na—2H2O | 0.26 | 225 | 58.5 |
| L-Valine | 0.5395 | 117 | 63.1215 |
| Linoleic acid | 0.000075 | 280 | 0.021 |
| Lipoic acid | | 206 | 0.078795 |
| Creatine | | | 500.00 |
| B-27 | | | 500 μl/L |
| Transferrin | | | 50 mg/L |

12. A culture medium for use in inducing the specification of neural stem cells (NSC) to the oligodendrocyte (OL) phenotype, the culture medium comprising:
Biotin
Calcium chloride
Choline chloride
Cupric sulfate
D-Calcium
pantothenate
D-Glucose
Ethanolamine
Ferric nitrate
Ferrous sulfate
Folic acid
Fumaric acid
Glycine
Hepes
Hydroxybutyrate
Hypoxanthine-Na
i-Inositol
L-Alanine
L-Arginine
hydrochloride
L-Asparagine-H2O
L-Aspartic acid
L-Cysteine-HCl—H2O
L-Cystine-2HCl
L-Glutamic Acid
L-Glutamine
L-Histidine-HCl—H2O
L-Isoleucine
L-Leucine
L-Lysine
hydrochloride
L-Methionine
L-Phenylalanine
L-Proline
L-Serine
L-Threonine
L-Tryptophan
L-Tyrosine-2Na-2H20
L-Valine
Linoleic acid
Lipoic acid
Magnesium chloride
Magnesium sulfate
Niacinamide
Phenol red
Potassium chloride
Putrescine-2HCl
Pyridoxine
hydrochloride
Pyruvic acid
Riboflavin
Sodium chloride
Sodium bicarbonate
Sodium phosphate,
mono.
Sodium phosphate,
dibas.
Sodium pyruvate
Thiamine
hydrochloride
Thymidine
Vitamin B12
Zinc sulfate
Creatine
B-27
bFGF
EGF
Kanamycin
Insulin
Transferrin
Pen/Strep

13. The culture medium of claim 12, wherein the culture medium comprises:

| | Molarity | Formula | |
|---|---|---|---|
| Components | (mM) | Weight | mg/L |
| Biotin | 0.000011075 | 244 | 0.002702 |
| Calcium chloride | 1.14975 | 111 | 127.6223 |
| Choline chloride | 0.0552 | 140 | 7.728 |
| Cupric sulfate | 0.0000038 | 250 | 0.00095 |
| D-Calcium | 0.00555 | 477 | 2.64735 |
| pantothenate | | | |
| D-Glucose | 17 | 180 | 3060 |
| D-(+)Galactose | 12.8 | | 2300 |
| Ethanolamine | 0.125 | 61 | 7.625 |
| Ferric nitrate | 0.000154 | 404 | 0.062216 |
| Ferrous sulfate | 0.001125 | 278 | 0.31275 |
| Folic acid | 0.00689 | 441 | 3.03849 |
| Fumaric acid | 0.25 | | 0 |
| Glycine | 0.26835 | 75 | 20.12625 |
| Hepes | 3.75 | | 0 |
| Hydroxybutyrate | 0.25 | | 0 |
| Hypoxanthine-Na | 0.01125 | | 0 |
| i-Inositol | 0.0625 | 180 | 11.25 |
| L-Alanine | 0.0375 | 89 | 3.3375 |
| L-Arginine | 0.624 | 211 | 131.664 |
| hydrochloride | | | |
| L-Asparagine-H2O | 0.0375 | 150 | 5.625 |
| L-Aspartic acid | 0.025 | 133 | 3.325 |
| L-Cysteine-HCl—H2O | 0.075 | 176 | 13.2 |
| L-Cystine-2HCl | 0.125 | 313 | 39.125 |
| L-Glutamic Acid | 0.025 | 147 | 3.675 |
| L-Glutamine | 2.25 | 146 | 328.5 |
| L-Histidine-HCl—H2O | 0.16245 | 210 | 34.1145 |
| L-Isoleucine | 0.5125 | 131 | 67.1375 |
| L-Leucine | 0.52075 | 131 | 68.21825 |
| L-Lysine | 0.5735 | 183 | 104.9505 |
| hydrochloride | | | |
| L-Methionine | 0.137 | 149 | 20.413 |
| L-Phenylalanine | 0.26125 | 165 | 43.10625 |
| L-Proline | 0.1125 | 115 | 12.9375 |
| L-Serine | 0.2875 | 105 | 30.1875 |
| L-Threonine | 0.17625 | 119 | 20.97375 |
| L-Tryptophan | 0.05255 | 204 | 10.7202 |
| L-Tyrosine-2Na—2H2O | 0.26 | 225 | 58.5 |
| L-Valine | 0.5395 | 117 | 63.1215 |
| Linoleic acid | 0.000075 | 280 | 0.021 |
| Lipoic acid | 0.00037025 | 206 | 0.076272 |
| Magnesium chloride | 0.225 | 95 | 21.375 |
| Magnesium sulfate | 0.50825 | 120 | 60.99 |
| Niacinamide | 0.0206 | 122 | 2.5132 |
| Phenol red | 0.01805 | 398 | 7.1839 |
| Potassium chloride | 4.435 | 75 | 332.625 |
| Putrescine-2HCl | 0.00037575 | 161 | 0.060496 |
| Pyridoxine | 0.0123 | 206 | 2.5338 |
| hydrochloride | | | |
| Pyruvic acid | 0.25 | | 0 |
| Riboflavin | 0.00070525 | 376 | 0.265174 |
| Sodium chloride | 118.085 | 58 | 6848.93 |
| Sodium bicarbonate | 36.375 | 84 | 3055.5 |
| Sodium phosphate, | 0.33975 | | 0 |
| mono. | | | |
| Sodium phosphate, | 0.125 | | 0 |
| dibas. | | | |
| Sodium pyruvate | 1.125 | 110 | 123.75 |
| Thiamine | 0.00775 | 337 | 2.61175 |
| hydrochloride | | | |
| Thymidine | 0.001125 | 242 | 0.27225 |
| Vitamin B12 | 0.000625 | 1355 | 0.846875 |
| Zinc sulfate | 0.001125 | 288 | 0.324 |
| Creatine hydrate | 3.355 | 131.13 | 01.75 |
| B-27 | 0.038194444 | | |
| bFGF | 0 | | |
| EGF | 0 | | |
| Kanamycin | 0 | | |
| Insulin | 0 | | 5 mg/L |
| Transferrin | | | 25 mg/L |
| Pen/Strep | 0 | | |

14. The culture medium of claim 12, wherein the culture medium comprises:

| | Molarity | Formula | |
|---|---|---|---|
| Components | (mM) | Weight | mg/L |
| Biotin | 0.000011075 | 244 | 0.002702 |
| Calcium chloride | | 111 | 137.3625 |
| Choline chloride | 0.0552 | 140 | 7.728 |
| Cupric sulfate | | 250 | 3735 |
| D-Calcium | 0.00555 | 477 | 2.64735 |
| pantothenate | | | |
| D-Glucose | 17 | 180 | 3060 |
| D-Galactose | | | 2300 |
| Ethanolamine | | 61 | 7.625 |
| Ferric nitrate | 0.000154 | 404 | 0.062216 |
| Ferrous sulfate | 0.001125 | 278 | 0.31275 |
| Folic acid | 0.00689 | 441 | 3.03849 |
| Fumaric acid | | | 34.5 |
| Glycine | | 75 | 30.9 |
| Hepes | | | 892.5 |
| Hydroxybutyrate | | | 40 |
| Hypoxanthine-Na | 0.01125 | | 1.78875 |
| i-Inositol | 0.0625 | 180 | 11.25 |
| L-Alanine | 0.0375 | 89 | 3.3375 |
| L-Arginine | 0.624 | 211 | 131.664 |
| hydrochloride | | | |
| L-Asparagine-H2O | 0.0375 | 150 | 5.625 |
| L-Aspartic acid | 0.025 | 133 | 3.325 |
| L-Cysteine-HCl—H2O | 0.075 | 176 | 13.2 |
| L-Cystine-2HCl | 0.125 | 313 | 39.125 |
| L-Glutamic Acid | 0.025 | 147 | 3.675 |
| L-Glutamine | 2.25 | 146 | 328.5 |
| L-Histidine-HCl—H2O | 0.16245 | 210 | 34.1145 |
| L-Isoleucine | 0.5125 | 131 | 67.1375 |
| L-Leucine | 0.52075 | 131 | 68.21825 |
| L-Lysine | 0.5735 | 183 | 104.9505 |
| hydrochloride | | | |
| L-Methionine | 0.137 | 149 | 20.413 |
| L-Phenylalanine | 0.26125 | 165 | 43.10625 |
| L-Proline | 0.1125 | 115 | 12.9375 |
| L-Serine | 0.2875 | 105 | 30.1875 |
| L-Threonine | | 119 | 42.39375 |
| L-Tryptophan | 0.05255 | 204 | 10.7202 |
| L-Tyrosine-2Na—2H2O | 0.26 | 225 | 58.5 |
| L-Valine | 0.5395 | 117 | 63.1215 |
| Linoleic acid | 0.000075 | 280 | 0.021 |
| Lipoic acid | | 206 | 0.078795 |
| Magnesium chloride | 0.225 | 95 | 21.375 |
| Magnesium sulfate | 0.50825 | 120 | 60.99 |
| Niacinamide | 0.0206 | 122 | 2.5132 |
| Phenol red | | 398 | 7.5023 |
| Potassium chloride | 4.435 | 75 | 332.625 |
| Putrescine-2HCl | 0.0512575 | 161 | 8.070246 |
| Pyridoxine | | 206 | 1.611195 |
| hydrochloride | | | |
| Pyruvic acid | | | 27.5 |
| Riboflavin | | 376 | 1.9796 |
| Sodium chloride | | 58 | 6846.465 |
| Sodium bicarbonate | | 84 | 2935 |
| Sodium phosphate, | | | 78.142 |
| mono. | | | |
| Sodium phosphate, | | | 53.256 |
| dibas. | | | |
| Sodium pyruvate | | 110 | 96.25 |
| Thiamine | 0.00775 | 337 | 2.61175 |
| hydrochloride | | | |
| Thymidine | 0.001125 | 242 | 0.27225 |
| Vitamin B12 | | 1355 | 0.508125 |
| Zinc sulfate | 0.001125 | 288 | 0.324 |
| Creatine | 3.355 | | 500 |
| B-27+ Retinoic acid | 0.038194444 | | 1 ml/L |
| bFGF | | | 10 ng/ml |
| EGF | | | 5 ng/ml |
| Kanamycin | | | 1 ml/L |
| Insulin | | | 7.5 mg/L |
| Sodium Selenite | | | 4 mg/L |
| Transferrin | | | 25 mg/L |
| Pen/Strep | | | 0.750 ml/L |
| Normocin | | | 1 ml/L |

15. The nutrient formula of claim 9, wherein the nutrient formula comprises one or more components selected from the group consisting of additional insulin, insulin-like growth factor-1 (IGF-1), insulin-like growth factor-2 (IGF-2).

16. The culture medium of claim 12, wherein the culture medium comprises additional transferrin.

17. A cell culture for inducing the specification of neural stem cells (NSC) to the oligodendrocyte (OL) phenotype, wherein the cell culture comprises:
neural stem cells; and
the nutrient formula of claim 9.

18. The nutrient formula of claim 1, wherein the stem cell is a neural stem cell.

19-21. (canceled)

22. A culture medium for use in inducing the specification of multipotent stem cells to oligodendrocyte progenitors, the culture medium comprising:
Biotin
Calcium chloride
Choline chloride
Cupric sulfate
D-Calcium
pantothenate
D-Glucose
D-Galactose
Ethanolamine
Ferric nitrate
Ferrous sulfate
Folic acid
Fumaric acid
Glycine
Hepes
Hydroxybutyrate
Hypoxanthine-Na
i-Inositol
L-Alanine
L-Arginine
hydrochloride
L-Asparagine-H2O
L-Aspartic acid
L-Cysteine-HCl—H2O
L-Cystine-2HCl
L-Glutamic Acid
L-Glutamine
L-Histidine-HCl—H2O
L-Isoleucine
L-Leucine
L-Lysine
hydrochloride
L-Methionine
L-Phenylalanine
L-Proline
L-Serine
L-Threonine
L-Tryptophan
L-Tyrosine-2Na—2H2O
L-Valine
Linoleic acid
Lipoic acid
Magnesium chloride
Magnesium sulfate
Niacinamide
Phenol red
Potassium chloride
Putrescine-2HCl
Pyridoxine
hydrochloride
Progesterone
Pyruvic acid
Riboflavin
Sodium chloride
Sodium bicarbonate
Sodium phosphate,
mono.
Sodium phosphate,
dibas.
Sodium pyruvate
Sodium selenite
Thiamine
hydrochloride
Thymidine
Vitamin B12
Zinc sulfate
Creatine
B-27
bFGF
EGF
Kanamycin
Insulin
Transferrin
Pen/Strep
Normocin

23. The culture medium of claim 22, wherein the culture medium comprises:

| | Molarity | Formula | |
|---|---|---|---|
| Components | (mM) | Weight | mg/L |
| Biotin | 0.000011075 | 244 | 0.002702 |
| Calcium chloride | 1.14975 | 111 | 127.6223 |
| Choline chloride | 0.0552 | 140 | 7.728 |
| Cupric sulfate | 0.0000038 | 250 | 0.00095 |
| D-Calcium | 0.00555 | 477 | 2.64735 |
| pantothenate | | | |
| D-Glucose | 17 | 180 | 3060 |
| D-Galactose | | | 4600 g/L |
| Ethanolamine | 0.125 | 61 | 7.625 |
| Ferric nitrate | 0.000154 | 404 | 0.062216 |
| Ferrous sulfate | 0.001125 | 278 | 0.31275 |
| Folic acid | 0.00689 | 441 | 3.03849 |
| Fumaric acid | 0.25 | | 0 |
| Glycine | 0.26835 | 75 | 20.12625 |
| Hepes | 3.75 | | 0 |
| Hydroxybutyrate | 0.25 | | 0 |
| Hypoxanthine-Na | 0.01125 | | 2.40 |
| i-Inositol | 0.0625 | 180 | 11.25 |
| L-Alanine | 0.0375 | 89 | 3.3375 |
| L-Arginine | 0.624 | 211 | 131.664 |
| hydrochloride | | | |
| L-Asparagine-H2O | 0.0375 | 150 | 5.625 |
| L-Aspartic acid | 0.025 | 133 | 3.325 |
| L-Cysteine-HCl—H2O | 0.075 | 176 | 13.2 |
| L-Cystine-2HCl | 0.125 | 313 | 39.125 |
| L-Glutamic Acid | 0.025 | 147 | 3.675 |
| L-Glutamine | 2.25 | 146 | 328.5 |
| L-Histidine-HCl—H2O | 0.16245 | 210 | 34.1145 |
| L-Isoleucine | 0.5125 | 131 | 67.1375 |
| L-Leucine | 0.52075 | 131 | 68.21825 |
| L-Lysine | 0.5735 | 183 | 104.9505 |
| hydrochloride | | | |
| L-Methionine | 0.137 | 149 | 20.413 |
| L-Phenylalanine | 0.26125 | 165 | 43.10625 |
| L-Proline | 0.1125 | 115 | 12.9375 |
| L-Serine | 0.2875 | 105 | 30.1875 |
| L-Threonine | 0.17625 | 119 | 20.97375 |
| L-Tryptophan | 0.05255 | 204 | 10.7202 |
| L-Tyrosine-2Na—2H2O | 0.26 | 225 | 58.5 |
| L-Valine | 0.5395 | 117 | 63.1215 |
| Linoleic acid | 0.000075 | 280 | 0.021 |
| Lipoic acid | 0.00037025 | 206 | 0.076272 |
| Magnesium chloride | 0.225 | 95 | 21.375 |
| Magnesium sulfate | 0.50825 | 120 | 60.99 |
| Niacinamide | 0.0206 | 122 | 2.5132 |
| Phenol red | 0.01805 | 398 | 7.1839 |
| Potassium chloride | 4.435 | 75 | 332.625 |
| Putrescine-2HCl | 0.00037575 | 161 | 17.26 |
| Pyridoxine | 0.0123 | 206 | 2.5338 |
| hydrochloride | | | |
| Progesterone | 25 nm | | |
| Pyruvic acid | 0.25 | | 0 |
| Riboflavin | 0.00070525 | 376 | 0.265174 |
| Sodium chloride | 118.085 | 58 | 6848.93 |
| Sodium bicarbonate | 36.375 | 84 | 5870 |
| Sodium phosphate, | 0.33975 | | 71 |
| mono. | | | |
| Sodium phosphate, | 0.125 | | 53.25 |
| dibas. | | | |
| Sodium pyruvate | 1.125 | 110 | 123.75 |
| Sodium selenite | | | 8 |
| Thiamine | 0.00775 | 337 | 2.61175 |
| hydrochloride | | | |
| Thymidine | 0.001125 | 242 | 0.27225 |
| Vitamin B12 | 0.000625 | 1355 | 0.846875 |
| Zinc sulfate | 0.001125 | 288 | 0.324 |
| Creatine | 3.355 | | 25/100 ml |
| B-27 | 0.038194444 | | 2 ml/L |
| bFGF | | | 0 |
| EGF | | | 0 |
| Kanamycin | | | 1 ml/L |
| Insulin | | | 10 mg/L |
| Transferrin | | | 150 mg/L |
| Pen/Strep | | | 0.5 ml/L |
| Normocin | | | 1 ml/L |

24-27. (canceled)
